(19) United States
(12) Patent Application Publication
(10) Pub. No.: US 2025/0078956 A1
(43) Pub. Date: Mar. 6, 2025

(54) Methods And Systems For Discovery Of Non-Embedded Target Genes

(71) Applicant: LifeMine Therapeutics, Inc., Cambridge, MA (US)

(72) Inventors: Michalis HADJITHOMAS, Cambridge, MA (US); Jinwoo KIM, Cambridge, MA (US); Sebastian THEOBALD, Cambridge, MA (US); Stephen Andrew WYKA, Cambridge, MA (US); Iain James MCFADYEN, Cambridge, MA (US); Gregory L. VERDINE, Cambridge, MA (US)

(21) Appl. No.: 18/660,474

(22) Filed: May 10, 2024

Related U.S. Application Data
(63) Continuation of application No. PCT/US2022/079965, filed on Nov. 15, 2022.
(60) Provisional application No. 63/264,150, filed on Nov. 16, 2021.

(51) Int. Cl. G16B 30/10; G16B 20/10; G16B 40/30

(52) U.S. Cl. CPC G16B 30/10; G16B 20/10; G16B 40/30

(57) ABSTRACT

The present disclosure relates to computer-based methods and systems for identifying non-embedded genes associated with biosynthetic gene clusters (BGCs), including non-embedded target genes (nETaGs) that are homologs of potential therapeutic targets, using comparative genomics techniques. Methods for identifying genes associated with (but not embedded within) biosynthetic gene clusters and applications thereof are described, including predicting the function of secondary metabolites based on the co-occurrence and/or co-evolution of genes encoding for secondary metabolites with biosynthetic gene clusters or their core enzymes, and prediction of biosynthetic gene clusters that produce secondary metabolites having an activity of interest.

### CROSS-REFERENCE TO RELATED APPLICATIONS

This application is a continuation of International Application No. PCT/US22/079965, which designated the United States and was filed on Nov. 15, 2022, published in English, which claims the priority benefit of U.S. Provisional Patent Application Ser. No. 63/264,150, filed Nov. 16, 2021, the contents of which are incorporated herein by reference in their entirety.

### FIELD

The present disclosure relates generally to methods and systems for identifying genes associated with (but not embedded within) biosynthetic gene clusters and applications thereof, including predicting the function of secondary metabolites based on the co-occurrence and/or co-evolution of genes encoding for secondary metabolites with biosynthetic gene clusters or their core enzymes, and prediction of biosynthetic gene clusters that produce secondary metabolites having an activity of interest.

### BACKGROUND

Microbes produce a wide variety of small molecule compounds, known as secondary metabolites or natural products, which have diverse chemical structures and functions. Some secondary metabolites allow microbes to survive adverse environments, while others serve as weapons of inter- and intra-species competition. See, e.g., Piel, J. Nat. Prod. Rep., 26:338-362, 2009. Many human medicines (including, e.g., antibacterial, antitumor agents, and insecticides) have been derived from secondary metabolites. See, e.g., Newman D. J. and Cragg G. M., J. Nat. Prod., 79:629-661, 2016.

Microbes synthesize secondary metabolites using enzyme proteins encoded by clusters of co-located genes called biosynthetic gene clusters (BGCs). Evidence is emerging that some microbial biosynthetic gene clusters contain genes that appear not to be involved in synthesis of the relevant biosynthetic products produced by the enzymes encoded by the clusters. In some cases, such non-biosynthetic genes have been described as “self-protective” because they encode proteins that apparently can render the host organism resistant to the relevant biosynthetic product. For example, in some cases, genes encoding transporters of the biosynthetic products, detoxification enzymes that act on the biosynthetic products, or resistant variants of proteins whose activities are targeted by the biosynthetic products, have been reported. See, for example, Cimermancic, et al., Cell 158:412, 2014; Keller, Nat. Chem. Biol. 11:671, 2015. Researchers have proposed that identification of such genes, and determination of their functions, could be useful in determining the role of the biosynthetic products synthesized by the enzymes of the clusters. See, for example, Ych, et al., ACS Chem. Biol. 11:2275, 2016; Tang, et al., ACS Chem. Biol. 10:2841, 2015; Regueira, et al., Appl, Environ. Microbiol. 77:3035, 2011; Kennedy, et al., Science 284:1368, 1999; Lowther, et al., Proc. Natl. Acad. Sci. USA 95:12153, 1998; Abe, et al., Mol. Genet. Genomics 268:130, 2002. United States Patent Application Publication No. US 2020/0211673 A1 provides insights that certain genes present in biosynthetic gene clusters, or located in close proximity to biosynthetic genes of the clusters (particularly in eukaryotic, e.g., fungal, biosynthetic gene clusters as contrasted with bacterial biosynthetic gene clusters) may represent homologs of human genes that are targets of therapeutic interest. Such genes, which are not involved in the synthesis of the secondary metabolite produced by the biosynthetic gene cluster, are referred to as “embedded target genes” (“ETaGs”) or “non-embedded target genes” (NETaGs) depending on whether or not they are located within the cluster of biosynthetic genes.

Traditionally, secondary metabolites have been identified from microbial cultures and screened for therapeutic activities against human targets of interest. However, the vast majority of microbes are not culturable, and even BGCs in culturable microbes can remain transcriptionally silent under laboratory conditions. Recent developments in nucleic acid and protein sequencing technologies and bioinformatics pipelines have enabled rapid identification of a large number of BGCs from environmental microbes without having to culture the microbes and test the bioactivity of the BGCs. See, e.g., Palazzotto E. and Weber T. Curr. Opin. Microbiol., 45:109-116, 2018. However, it remains a challenge to precisely define the genomic boundaries of BGCs using pure computational methods. There are also no computational pipelines available to identify genes associated with (but not embedded within) biosynthetic gene clusters, or for predicting the function of secondary metabolites and predicting biosynthetic gene clusters that produce secondary metabolites having an activity of interest.

### SUMMARY

Disclosed herein are exemplary methods and systems for identifying resistance genes (e.g., embedded target genes (ETaGs) and/or non-embedded target genes (NETaGs)) associated with a core synthase or other gene from a biosynthetic gene cluster (BGC) that encodes a biosynthetic pathway for secondary metabolites. The described methods and systems may also be used for, e.g., predicting the function of secondary metabolites based on the co-occurrence and/or co-evolution of resistance genes (e.g., ETaGs or NETaGs) with the genes of biosynthetic gene clusters, and prediction of biosynthetic gene clusters that produce secondary metabolites having an activity of interest.

Disclosed herein are computer-implemented methods for identifying resistance genes comprising: receiving a selection of at least one target sequence of interest; receiving a selection of target genomes from a genomics database, wherein the selection of target genomes comprises a plurality of target genomes from organisms that are known to produce, or are likely to produce, secondary metabolites; performing a search to identify homologs of the at least one target sequence in the plurality of target genomes; generating a phylogenetic tree based on the identified homologs of the at least one target sequence; classifying the genomes of the plurality of target genomes as positive genomes or negative genomes based on the phylogenetic tree, wherein positive genomes are genomes that belong to a clade for which multiple copies of the at least one target sequence homolog are present, wherein negative genomes are genomes that belong to a clade for which a single copy of the at least one target sequence homolog is present; and wherein a target sequence homolog that is present in multiple copies in a positive genome is a putative resistance gene; determining, based at least in part on the classification of positive and negative genomes, at least one genomic parameter selected from the following: i) one or more scores indicative of co-occurrence of the at least one target sequence homolog (putative resistance gene) and one or more genes associated with a biosynthetic gene cluster (BGC); ii) one or more scores indicative of co-evolution of the at least one target sequence homolog (putative resistance gene) and one or more genes associated with a BGC; iii) one or more scores indicative of co-regulation of the at least one target sequence homolog (putative resistance gene) with one or more genes associated with a BGC; and iv) one or more scores indicative of co-expression of the at least one target sequence homolog (putative resistance gene) with one or more genes associated with a BGC; and determining, based on the at least one genomic parameter, a likelihood that the putative resistance gene is a resistance gene.

In some embodiments, determining the likelihood that the putative NETaG is a non-embedded target gene (NETaG) comprises comparing the at least one determined genomic parameter to at least one predetermined threshold.

In some embodiments, the selection of at least one target sequence of interest is provided as input by a user of a system configured to perform the computer-implemented method. In some embodiments, the at least one target sequence of interest comprises an amino acid sequence, a nucleotide sequence, or any combination thereof. In some embodiments, the at least one target sequence of interest comprises a peptide sequence or portion thereof, a protein sequence or portion thereof, a protein domain sequence or portion thereof, a gene sequence or portion thereof, or any combination thereof. In some embodiments, the at least one target sequence of interest comprises a mammalian sequence, a human sequence, a plant sequence, a fungal sequence, a bacterial sequence, an archaea sequence, a viral sequence, or any combination thereof.

In some embodiments, the at least one target sequence of interest comprises a primary target sequence and one or more related sequences. In some embodiments, the one or more related sequences comprise sequences that are functionally-related to the primary target sequence. In some embodiments, the one or more related sequences comprise sequences that are pathway-related to the primary target sequence.

In some embodiments, the selection of target genomes is provided as input by a user of a system configured to perform the computer-implemented method. In some embodiments, the plurality of target genomes comprise plant genomes, fungal genomes, bacterial genomes, or any combination thereof. In some embodiments, the genomics database comprises a public genomics database. In some embodiments, the genomics database comprises a proprietary genomics database.

In some embodiments, the search to identify homologs of the at least one target sequence comprises identification of homologs based on probabilistic sequence alignment models. In some embodiments, the probabilistic sequence alignment models are profile hidden Markov models (pHMMs). In some embodiments, homologs are identified based on a comparison of probabilistic sequence alignment model scores to a predefined threshold.

In some embodiments, the search to identify homologs of the at least one target sequence comprises identification of homologs based on alignment of sequences using a local sequence alignment search tool, calculation of a sequence homology metrics based on the alignments, and comparison of the calculated sequence homology metrics to a predefined threshold. In some embodiments, the local sequence alignment search tool comprises BLAST, DIAMOND, HMMER, Exonerate, or ggsearch. In some embodiments, the predefined threshold comprises a threshold for percent sequence identity, percent sequence coverage, E-value, or bitscore value.

In some embodiments, the search to identify homologs of the at least one target sequence comprises identification of homologs based on use of a gene and/or protein domain annotation tool. In some embodiments, the gene and/or protein domain annotation tool comprises InterProScan or EggNOG.

In some embodiments, the generation of phylogenetic trees based on the identified homologs of the at least one target sequence comprises alignment of homolog sequences using an alignment software tool, trimming of the aligned homolog sequences using a sequence trimming software tool, and construction of a phylogenetic tree using phylogenetic tree building software tool. In some embodiments, the alignment software tool comprises MAFFT, MUSCLE, or ClustalW. In some embodiments, the sequence trimming software tool comprises trimAI, GBlocks, or ClipKIT. In some embodiments, the phylogenetic tree building software tool comprises FastTree, IQ-TREE, RAXML, MEGA, MrBayes, BEAST, or PAUP. In some embodiments, the construction of the phylogenetic tree is based on a maximum likelihood algorithm, parsimony algorithm, neighbor joining algorithm, distance matrix algorithm, or Bayesian inference algorithm.

In some embodiments, the one or more scores indicative of co-occurrence are determined based on identifying positive correlations between the presence of multiple copies of a putative resistance gene and the presence of the one or more genes of a BGC in positive genomes. In some embodiments, identifying the positive correlations between the presence of multiple copies of the putative resistance gene and the presence of the one or more genes of a BGC in positive genomes comprises the use of a clustering algorithm to cluster aligned protein sequences, aligned nucleotide sequences, aligned protein domain sequences, or aligned pHMMs for a group of BGCs to identify BGC communities within the plurality of target genomes. In some embodiments, identifying the positive correlations between the presence of multiple copies of the putative resistance gene and the presence of the one or more genes of a BGC in positive genomes comprises the use of a phylogenetic analysis of protein sequences or protein domains for a group of BGCs to identify BGC communities within the plurality of target genomes. In some embodiments, identifying the positive correlations between the presence of multiple copies of the putative resistance gene and the presence of the one or more genes of a BGC in positive genomes comprises choosing genomes with a specific taxonomy to identify BGC communities within the plurality of target genomes.

In some embodiments, the one or more scores indicative of co-evolution of a putative resistance gene and the one or more genes associated with a BGC are determined based on a co-evolution correlation score, a co-evolution rank score, a co-evolution slope score, or any combination thereof. In some embodiments, the co-evolution correlation score is based on a correlation between pairwise percent sequence identities of a cluster of orthologous groups (COG) for the putative resistance gene and pairwise percent sequence identities of a cluster of orthologous groups (COG) for one of the one or more genes associated with a BGC. In some embodiments, the co-evolution rank score is based on a ranking of a correlation coefficient of a COG that contains one of the one or more genes associated with a BGC in ascending order in relation to a COG that contains the putative resistance gene. In some embodiments, in the case of ties for a distance score, the rank for all COGs in the tie is set equal to a lowest rank in the group. In some embodiments, the co-evolution slope score is based on an orthogonal regression of pairwise percent sequence identities of a COG for the putative resistance gene and pairwise percent sequence identities of a COG for one of the one or more genes associated with a BGC. In some embodiments, only COGs arising from unique positive genomes that have more than three genes remaining after removing corresponding genes from negative genomes are used to evaluate a co-evolution correlation score, a co-evolution rank score, or a co-evolution slope score.

In some embodiments, the one or more scores indicative of co-regulation are based on DNA motif detection from intergenic sequences of the one or more genes associated with a BGC and the putative resistance gene.

In some embodiments, the one or more scores indicative of co-expression are based on a differential expression analysis and/or a clustering analysis of global transcriptomics data.

In some embodiments, the one or more genes associated with a biosynthetic gene cluster (BGC) comprise an anchor gene, a core synthase gene, a biosynthetic gene, a gene not involved in the biosynthesis of a secondary metabolite produced by the BGC, or any combination thereof.

In some embodiments, the putative resistance gene is a putative embedded target gene (pETaG) or a putative non-embedded target gene (pNETaG).

In some embodiments, the resistance gene is an embedded target gene (ETaG) or a non-embedded target gene (NETaG).

Also disclosed herein are computer-implemented methods for predicting a function of a secondary metabolite comprising: receiving a selection of at least one target sequence of interest, wherein the at least one target sequence of interest corresponds to a gene sequence associated with a biosynthetic gene cluster (BGC) known to produce the secondary metabolite; receiving a selection of target genomes from a genomics database, wherein the selection of target genomes comprises a plurality of target genomes from organisms that are known to produce secondary metabolites; performing a search to identify homologs of the at least one target sequence in the plurality of target genomes; generating a phylogenetic tree based on the identified homologs of the at least one target sequence; classifying the genomes of the plurality of target genomes as positive genomes or negative genomes based on the phylogenetic tree, wherein positive genomes are genomes that belong to a clade for which multiple copies of the at least one target sequence homolog are present, wherein negative genomes are genomes that belong to a clade for which a single copy of the at least one target sequence homolog is present; and wherein a target sequence homolog that is present in multiple copies in a positive genome is a putative resistance gene; determining, based at least in part on the classification of positive and negative genomes, at least one genomic parameter selected from the following: i) one or more scores indicative of co-occurrence of the at least one target sequence homolog (putative resistance gene) and one or more genes associated with the BGC; ii) one or more scores indicative of co-evolution of the at least one target sequence homolog (putative resistance gene) and one or more genes associated with the BGC; iii) one or more scores indicative of co-regulation of the at least one target sequence homolog (putative resistance gene) with one or more genes associated with the BGC; and iv) one or more scores indicative of co-expression of the at least one target sequence homolog (putative resistance gene) with one or more genes associated with the BGC; and determining, based on the at least one genomic parameter, a likelihood that the putative resistance gene is a resistance gene that encodes a protein target that is acted upon by the secondary metabolite.

In some embodiments, determining the likelihood that the putative resistance gene is a resistance gene that encodes a protein target that is acted upon by the secondary metabolite comprises comparing the at least one determined genomic parameter to at least one predetermined threshold.

In some embodiments, the selection of at least one target sequence of interest is provided as input by a user of a system configured to perform the computer-implemented method. In some embodiments, the at least one target sequence of interest comprises an amino acid sequence, a nucleotide sequence, or any combination thereof. In some embodiments, the at least one target sequence of interest comprises a peptide sequence or portion thereof, a protein sequence or portion thereof, a protein domain sequence or portion thereof, a gene sequence or portion thereof, or any combination thereof.

In some embodiments, the selection of target genomes is provided as input by a user of a system configured to perform the computer-implemented method. In some embodiments, the plurality of target genomes comprise plant genomes, fungal genomes, bacterial genomes, or any combination thereof. In some embodiments, the genomics database comprises a public genomics database or a proprietary genomics database.

In some embodiments, the search to identify homologs of the at least one target sequence comprises identification of homologs based on probabilistic sequence alignment models. In some embodiments, the probabilistic sequence alignment models are profile hidden Markov models (pHMMs). In some embodiments, homologs are identified based on a comparison of probabilistic sequence alignment model scores to a predefined threshold.

In some embodiments, the search to identify homologs of the at least one target sequence comprises identification of homologs based on alignment of sequences using a local sequence alignment search tool, calculation of a sequence homology metrics based on the alignments, and comparison of the calculated sequence homology metrics to a predefined threshold. In some embodiments, the predefined threshold comprises a threshold for percent sequence identity, percent sequence coverage, E-value, or bitscore value.

In some embodiments, the search to identify homologs of the at least one target sequence comprises identification of homologs based on use of a gene and/or protein domain annotation tool.

In some embodiments, the generation of phylogenetic trees based on the identified homologs of the at least one target sequence comprises alignment of homolog sequences using an alignment software tool, trimming of the aligned homolog sequences using a sequence trimming software tool, and construction of a phylogenetic tree using phylogenetic tree building software tool.

In some embodiments, the at least one target sequence of interest comprises a known NETaG sequence or core synthase gene sequence.

Disclosed herein are computer-implemented methods for identifying a biosynthetic gene cluster (BGC) that encodes biosynthetic enzymes for producing a secondary metabolite having an activity of interest, the methods comprising: receiving a selection of at least one target sequence of interest, wherein the at least one target sequence of interest comprises a sequence that encodes a therapeutic target of interest; receiving a selection of target genomes from a genomics database, wherein the selection of target genomes comprises a plurality of target genomes from organisms that are known to produce secondary metabolites; performing a search to identify homologs of the at least one target sequence in the plurality of target genomes; generating a phylogenetic tree based on the identified homologs of the at least one target sequence; classifying the genomes of the plurality of target genomes as positive genomes or negative genomes based on the phylogenetic tree, wherein positive genomes are genomes that belong to a clade for which multiple copies of the at least one target sequence homolog are present, wherein negative genomes are genomes that belong to a clade for which a single copy of the at least one target sequence homolog is present; and wherein a target sequence homolog that is present in multiple copies in a positive genome is a putative resistance gene; determining, based at least in part on the classification of positive and negative genomes, at least one genomic parameter selected from the following: i) one or more scores indicative of co-occurrence of the at least one target sequence homolog (putative resistance gene) and one or more genes associated with a biosynthetic gene cluster (BGC); ii) one or more scores indicative of co-evolution of the at least one target sequence homolog (putative resistance) and one or more genes associated with a BGC; iii) one or more scores indicative of co-regulation of the at least one target sequence homolog (putative resistance gene) with one or more genes associated with a BGC; and iv) one or more scores indicative of co-expression of the at least one target sequence homolog (putative resistance gene) with one or more genes associated with a BGC; and determining, based on the at least one genomic parameter, a likelihood that the putative resistance gene is an actual resistance gene associated with a BGC that produces a secondary metabolite that acts upon a protein product encoded by the resistance gene.

In some embodiments, determining the likelihood that the putative resistance gene is an actual resistance gene associated with the BGC that produces the secondary metabolite comprises comparing the at least one determined genomic parameter to at least one predetermined threshold.

In some embodiments, the selection of at least one target sequence of interest is provided as input by a user of a system configured to perform the computer-implemented method. In some embodiments, the at least one target sequence of interest comprises an amino acid sequence, a nucleotide sequence, or any combination thereof. In some embodiments, the at least one target sequence of interest comprises a peptide sequence or portion thereof, a protein sequence or portion thereof, a protein domain sequence or portion thereof, a gene sequence or portion thereof, or any combination thereof.

In some embodiments, the selection of target genomes is provided as input by a user of a system configured to perform the computer-implemented method. In some embodiments, the plurality of target genomes comprise plant genomes, fungal genomes, bacterial genomes, or any combination thereof. In some embodiments, the genomics database comprises a public genomics database or a proprietary genomics database.

In some embodiments, the search to identify homologs of the at least one target sequence comprises identification of homologs based on probabilistic sequence alignment models. In some embodiments, the probabilistic sequence alignment models are profile hidden Markov models (pHMMs). In some embodiments, homologs are identified based on a comparison of probabilistic sequence alignment model scores to a predefined threshold.

In some embodiments, the search to identify homologs of the at least one target sequence comprises identification of homologs based on alignment of sequences using a local sequence alignment search tool, calculation of a sequence homology metrics based on the alignments, and comparison of the calculated sequence homology metrics to a predefined threshold. In some embodiments, the predefined threshold comprises a threshold for percent sequence identity, percent sequence coverage, E-value, or bitscore value.

In some embodiments, the search to identify homologs of the at least one target sequence comprises identification of homologs based on use of a gene and/or protein domain annotation tool.

In some embodiments, the generation of phylogenetic trees based on the identified homologs of the at least one target sequence comprises alignment of homolog sequences using an alignment software tool, trimming of the aligned homolog sequences using a sequence trimming software tool, and construction of a phylogenetic tree using phylogenetic tree building software tool.

In some embodiments, the computer-implemented method further comprises performing an in vitro assay to test a secondary metabolite produced by the identified BGC for activity against the therapeutic target of interest.

In some embodiments, the computer-implemented method further comprises performing an in vivo assay to test a secondary metabolite produced by the identified BGC for activity against the therapeutic target of interest.

Also disclosed herein are systems comprising: one or more processors; and a memory communicatively coupled to the one or more processors and configured to store instructions that, when executed by the one or more processors, cause the system to perform any of the methods described herein.

Disclosed herein are non-transitory computer-readable storage media storing one or more programs, the one or more programs comprising instructions which, when executed by one or more processors of a system, cause the system to perform any of the methods described herein.

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein.

### INCORPORATION BY REFERENCE

All publications, patents, and patent applications mentioned in this specification are herein incorporated by reference in their entirety to the same extent as if each individual publication, patent, or patent application was specifically and individually indicated to be incorporated by reference in its entirety. In the event of a conflict between a term herein and a term in an incorporated reference, the term herein controls.

### BRIEF DESCRIPTION OF THE FIGURES

Various aspects of the disclosed methods, devices, and systems are set forth with particularity in the appended claims. A better understanding of the features and advantages of the disclosed methods, devices, and systems will be obtained by reference to the following detailed description of illustrative embodiments and the accompanying drawings, of which:

FIG. 1 provides a non-limiting example of a process flowchart for identifying putative resistance genes (e.g., putative embedded target genes (pETaGs) and/or putative non-embedded target genes (pNETaGs)) and evaluating their likelihood of being actual resistance genes (e.g., EtaGs and/or NETaGs).

FIG. 2 provides a non-limiting schematic illustration of a computing device in accordance with one or more examples of the disclosure.

FIG. 3 provides a non-limiting example of a maximum likelihood phylogenetic tree of succinate dehydrogenase complex subunit C (SDHC) homologs.

FIG. 4 provides an exemplary illustration of a gene cluster comparison plot.

### DETAILED DESCRIPTION

Disclosed herein are exemplary methods and systems for identifying resistance genes (e.g., embedded target genes (ETaGs) and/or non-embedded target genes (NETaGs)) associated with a core synthase or other gene from a biosynthetic gene cluster (BGC) that encodes a biosynthetic pathway for secondary metabolites. The described methods and systems may also be used for, e.g., predicting the function of secondary metabolites based on the co-occurrence and/or co-evolution of resistance genes (e.g., ETaGs or NETaGs) with the genes of biosynthetic gene clusters, and prediction of biosynthetic gene clusters that produce secondary metabolites having an activity of interest.

In some instances, for example, the disclosed methods may comprise: receiving a selection of at least one target sequence of interest; receiving a selection of target genomes from a genomics database, wherein the selection of target genomes comprises a plurality of target genomes from organisms that are known to produce, or are likely to produce, secondary metabolites; performing a search to identify homologs of the at least one target sequence in the plurality of target genomes; generating a phylogenetic tree based on the identified homologs of the at least one target sequence; classifying the genomes of the plurality of target genomes as positive genomes or negative genomes based on the phylogenetic tree, wherein positive genomes are genomes that belong to a clade for which multiple copies of the at least one target sequence homolog are present, wherein negative genomes are genomes that belong to a clade for which a single copy of the at least one target sequence homolog is present; and wherein a target sequence homolog that is present in multiple copies in a positive genome is a putative NETaG; determining, based at least in part on the classification of positive and negative genomes, at least one genomic parameter selected from the following: i) one or more scores indicative of co-occurrence of the at least one target sequence homolog (putative NETaG) and one or more genes associated with a biosynthetic gene cluster (BGC); ii) one or more scores indicative of co-evolution of the at least one target sequence homolog (putative NETaG) and one or more genes associated with a BGC; iii) one or more scores indicative of co-regulation of the at least one target sequence homolog (putative NETaG) with one or more genes associated with a BGC; and iv) one or more scores indicative of co-expression of the at least one target sequence homolog (putative NETaG) with one or more genes associated with a BGC; and determining, based on the at least one genomic parameter, a likelihood that the putative NETaG is a non-embedded target gene (NETaG).

### Definitions

Unless otherwise defined, all of the technical terms used herein have the same meaning as commonly understood by one of ordinary skill in the art in the field to which this disclosure belongs.

As used in this specification and the appended claims, the singular forms “a”, “an”, and “the” include plural references unless the context clearly indicates otherwise. Any reference to “or” herein is intended to encompass “and/or” unless otherwise stated, and encompasses any and all possible combinations of one or more of the associated listed items.

As used herein, the terms “includes, “including,” “comprises,” and/or “comprising” specify the presence of stated features, integers, steps, operations, elements, components, and/or units but do not preclude the presence or addition of one or more other features, integers, steps, operations, elements, components, units, and/or groups thereof.

As used herein, the term “about” a number refers to that number plus or minus 10% of that number. The term ‘about’ when used in the context of a range refers to that range minus 10% of its lowest value and plus 10% of its greatest value.

As used herein, a “secondary metabolite” refers to an organic small molecule compound produced by archaea, bacteria, fungi or plants, which is not directly involved in the normal growth, development, or reproduction of the host organism, but is required for interaction of the host organism with its environment. Secondary metabolites are also known as natural products or genetically encoded small molecules. The term “secondary metabolite” is used interchangeably herein with “biosynthetic product” when referring to the product of a biosynthetic gene cluster.

The terms “biosynthetic gene cluster” or “BGC” are used herein interchangeably to refer to a locally clustered group of one or more genes that together encode a biosynthetic pathway for the production of a secondary metabolite. Exemplary BGCs include, but are not limited to, biosynthetic gene clusters for the synthesis of non-ribosomal peptide synthetases (NRPS), polyketide synthases (PKS), terpenes, and bacteriocins. See, for example, Keller N, “Fungal secondary metabolism: regulation, function and drug discovery.” Nature Reviews Microbiology 17.3 (2019): 167-180 and Fischbach M. and Voigt C.A., PROKARYOTIC GENE CLUSTERS: A RICH TOOLBOX FOR SYNTHETIC BIOLOGY. In: Institute of Medicine (US) Forum on Microbial Threats. The Science and Applications of Synthetic and Systems Biology: Workshop Summary. Washington (DC): National Academies Press (US); 2011. A21. BGCs contain genes encoding signature biosynthetic proteins that are characteristic of each type of BGC. The longest biosynthetic gene in a BGC is referred to herein as the “core synthase gene” of a BGC. In addition to genes involved in the biosynthesis of a secondary metabolite, a BGC may also include other genes, e.g., genes that encode products that are not involved in the biosynthesis of a secondary metabolite, which are interspersed among the biosynthetic genes. These genes are referred to herein as being “associated” with the BGC if their products are functionally related to the secondary metabolite of the BGC. Some genes, e.g., genes not involved in the biosynthesis of a secondary metabolite produced by a BGC, are referred to herein as being “embedded” in the BGC if their products are functionally related to the secondary metabolite of the BGC and they are physically located in close proximity to the biosynthetic genes of the cluster. Some genes, e.g., genes not involved in the biosynthesis of a secondary metabolite produced by a BGC, are referred to herein as “non-embedded” if their products are functionally related to the secondary metabolite of a BGC but they are not physically located in close proximity to the biosynthetic genes of the BGC. An “anchor gene” refers to a biosynthetic gene or a gene that is not involved in the biosynthesis of a secondary metabolite produced by a BGC that is co-localized with a BGC and is known to be functionally related (i.e., associated) with the BGC.

The term “co-localize” refers to presence of two or more genes in close spatial positions, such as no more than about 200 kb, no more than about 100 kb, no more than about 50 kb, no more than about 40 kb, no more than about 30 kb, no more than about 20 kb, no more than about 10 kb, no more than about 5 kb, or less, in a genome.

The term “homolog” refers to a gene that is part of a group of genes that are related by descent from a common ancestor (i.e., the gene sequences (i.e., nucleic acid sequences) of the group of genes and/or the sequences of their protein products are inherited through a common origin. Homologs may arise through speciation events (giving rise to “orthologs”), through gene duplication events, or through horizontal gene transfer events. Homologs may be identified by phylogenetic methods, through identification of common functional domains in the aligned nucleic acid or protein sequences, or through sequence comparisons.

The term “ortholog” refers to a gene that is part of a group of genes that are predicted to have evolved from a common ancestral gene by speciation.

The terms “bidirectional best hit” and “BBH” are used herein interchangeably to refer to the relationship between a pair of genes in two genomes (i.e., a first gene in a first genome and a second gene in a second genome) wherein the first gene or its protein product has been identified as having the most similar sequence in the first genome as compared to the second gene or its protein product in the second genome, and wherein the second gene or its protein product has been identified as having the most similar sequence in the second genome as compared to the first gene or its protein product in the first genome. The first gene is the bidirectional best hit (BBH) of the second gene, and the second gene is the bidirectional best hit (BBH) of the first gene. BBH is a commonly used method to infer orthology.

As used herein, “sequence similarity” between two genes means similarity of either the nucleic acid (e.g., mRNA) sequences encoded by the genes or the amino acid sequences of the gene products.

“Percent (%) sequence identity” or “percent (%) sequence homology” with respect to the nucleic acid sequences (or protein sequences) described herein is defined as the percentage of nucleotide residues (or amino acid residues) in a candidate sequence that are identical or homologous with the nucleotide residues (or amino acid residues) in the oligonucleotide (or polypeptide) with which a candidate sequence is being compared, after aligning the sequences and considering any conservative substitutions as part of the sequence identity. Homology between different amino acid residues in a polypeptide is determined based on a substitution matrix, such as the BLOSUM (BLOcks SUbstitution Matrix). Methods for aligning sequences and determining percent sequence identity or percent sequence homology for nucleic acid or protein sequences are well known to those of the skill in the art. Examples of publicly available computer software that may be used include, but are not limited to, BLAST (Basic Local Alignment Search Tool; software for comparing the amino-acid sequences of proteins or the nucleotide sequences of DNA and/or RNA molecules), BLAST-2, ALIGN or Megalign (DNASTAR) software. Any of a variety of suitable parameters for measuring sequence alignment and determining percent sequence identity or homology may be determined by those of skill in the art, including use of algorithms required to achieve maximal alignment over the full length of the sequences being compared.

Certain aspects of the present disclosure include process steps and instructions described herein in the form of an algorithm. It should be noted that the process steps and instructions of the present disclosure could be embodied in software, firmware, or hardware and, when embodied in software, could be downloaded to reside on and be operated from different platforms used by a variety of operating systems. Unless specifically stated otherwise as apparent from the following discussion, it is appreciated that, throughout the description, discussions utilizing terms such as “processing,” “computing,” “calculating,” “determining,” “displaying,” “generating” or the like, refer to the action and processes of a computer system, or similar electronic computing device, that manipulates and transforms data represented as physical (electronic) quantities within the computer system memories or registers or other such information storage, transmission, or display devices.

The section headings used herein are for organizational purposes only and are not to be construed as limiting the subject matter described. The description is presented to enable one of ordinary skill in the art to make and use the invention, and is provided in the context of a patent application and its requirements.

Methods for Identifying Resistance Genes (e.g., Embedded Target Genes and/or Non-Embedded Target Genes)

Microbes synthesize secondary metabolites (SMs) using enzymes encoded by clusters of co-located genes called biosynthetic gene clusters (BGCs). Many SMs target primary metabolism enzymes, therefore the BGC may contain a so called “resistance copy” of a primary metabolism gene that has been described as “self-protective” because they encode proteins that may render the host organism resistant to the SM produced by the BGC. This “resistance copy” (or “resistance gene”) can be referred to as an “embedded target gene” or “ETaG” if it is located in close proximity to one of the biosynthetic genes of the BGC, or can be referred to as a “non-embedded target gene” or “NETaG” if is not located in close proximity to one of the biosynthetic genes of the BGC. The identification of ETaGs and NETaGs could be useful in determining the role of the SMs synthesized by the enzymes of the clusters. Methods for the identification of ETaGs have been described in co-pending International Patent Application Nos. PCT/US2022/049016 and PCT/US2022/049040, the contents of each of which are incorporated herein by reference in their entireties.

Current BGC prediction tools predict regions in the genome that constitute a BGC (see the section entitled “BGC annotation” below for details). Then, by identifying ETaGs within the genomic boundaries of a biosynthetic cluster, one can predict the potential targets of the SMs synthesized by the BGCs. One example is the 3-hydroxy-3-methylglutaryl-CoA (HMG-CoA) gene ETaG that is located within the Lovastatin gene cluster and confers resistance to Lovastatin. Another example is the Inosine-5′-monophosphate dehydrogenase (IMPDH) ETaG (gene name: mpaF) located within the Mycophenolic acid gene cluster that confers resistance to Mycophenolic acid. Since this approach relies on ETaGs being embedded within or positioned in proximity to the BGC to predict the function of the BGC, it fails to predict functions for BGCs that do not include ETaGs.

The present disclosure describes (i) location-independent methods for identifying non-embedded target genes (NETaGs), (ii) methods for predicting the function of SMs by correlation and/or coevolution of BGCs and/or core enzymes with NETaGs and (iii) methods for predicting the BGC responsible for production of an SM with an activity of interest. The methods described herein are superior to previous approaches since they enable detection of BGCs of interest and prediction of their functions independently of the location of target genes.

Targets of interest: The methods described herein can be performed using targets of interest (or target sequences of interest) that can be any amino acid sequences or nucleotide sequences from genomes of any type of organism including, but not limited to, mammalian genomes, human genomes, avian genomes, reptilian genomes, plant genomes, fungal genomes, bacterial genomes, archaca genomes, viral genomes, etc. Targets of interest may comprise any type of biological sequence such as gene sequences or portions thereof, protein sequences or portions thereof, protein domain sequences or portions thereof, peptide sequences or portions thereof, etc.

Target genome selection: The methods described herein are suitable for identifying ETaGs, NETaGs, and/or BGCs in any type of target genome containing BGCs or genomes for organisms that are known to produce secondary metabolites. Bacterial, plant, and fungal genomes are known to encode biosynthetic gene clusters. Without wishing to be bound by any theory or hypothesis, fungal genomes are eukaryotic genomes that are phylogenetically more related to mammalian genomes than bacterial or plant genomes. Thus, fungal genomes may be preferred for the identification of ETaGs or NETaGs that are homologous to human target genes and that encode the targets of the secondary metabolites produced by the BGCs.

Target homolog search: Protein or DNA homologs for a target sequence within given genomes can be detected using, for example:
1) Probabilistic sequence alignment models, including, e.g., profile hidden Markov models (pHMMs), by comparing probabilistic model scores to one or more predetermined thresholds (e.g., a trusted cutoff threshold). In some instances, such predetermined thresholds may be determined based on, e.g., the lowest bitscore for known homologs.
2) Sequence alignment tools, including, e.g., BLAST (basic local alignment search tool), DIAMOND, HMMER, Exonerate, or ggsearch, by comparing sequence alignment or sequence homology metrics such as percent sequence identity, percent sequence coverage, E-value, or bitscore, etc., to one or more predetermined thresholds.
3) Gene sequence/protein domain annotation tools such as InterProScan or EggNOG.

Phylogenetic tree creation based on the identified target homologs: Homologs of the target sequence(s) identified as a result of performing a target homology search may be used to generate phylogenetic trees for the selected target genomes. To determine phylogenetic distances, the protein or DNA homologs of the target(s) can be individually aligned using any alignment software (such as MAFFT, MUSCLE, or ClustalW, etc.), and trimmed using any sequence trimming software (e.g., trimAI, GBlocks, or ClipKIT) to remove gaps, following which multiple sequence alignments can be performed using any phylogenetic tree building software known to those of skill in the art (e.g., FastTree, IQ-TREE, RAXML, MEGA, MrBayes, BEAST, or PAUP) to provide a phylogenetic tree of the homologous sequences (e.g., homologous gene sequences or protein sequences). Phylogenetic trees can be constructed using any of a variety of different algorithms known to those of skill in the art including, but not limited to, maximum likelihood algorithms, parsimony algorithms, neighbor joining algorithms, distance matrix algorithms, or Bayesian inference algorithms.

Differentiation of house copies and additional copies of candidate NETaGs: From the phylogenetic tree, two groups (clades) of genomes comprising homologs of the target sequence(s) (e.g., target genes) may be identified. One clade contains genomes that comprise a single copy of a target gene homolog(s), indicating that the homologs are the “house-copy” of the gene (i.e., the single copy of target gene homolog(s) present in organisms of the first clade are assumed to have a house-keeping function only). The other clade contains genomes that comprise additional copies of the target gene homologs, which are required for the normal functioning of primary metabolism in the presence of a BGC product (i.e., the multiple copies of a target gene homolog in organisms of the second clade are assumed to be potential resistance-related genes due to their increased copy number). Target gene homologs which are present in multiple copies may thus be the candidate (or putative) NETaGs.

In some instances, other targets can be examined that may be related or correlated to the primary target of interest. These relationships could be functional relationships (e.g., genes that share similar function) or pathway relationships (e.g., genes that are members of the same pathway). For example, if KRAS is a primary target, one can also examine the copy number variation for additional RAS homologs such as HRAS, NRAS, MRAS, ERAS, RRAS2, RRAS, etc. In addition, one can also examine the copy number variation of genes within the RAS pathway, such as RAS-GEF, RAS-GAP, RAF, MEK, ERK, PI3K, PDK1, AKT, etc. Genomes with higher copy numbers of genes that are functionally-related or pathway-related to the primary target may thus harbor additional candidate NETaGs in the form of the functionally-related or pathway-related genes.

Positive and negative genome classification: Genomes are classified based on the number of target homologs, or genes related to the target homologs, that are encoded therein. In genomes that encode multiple copies of target homologs, one of the copies is assumed to have resistance to the specific BGC product and is thus required for primary metabolism to function when the BGC product is present. Genomes containing multiple copies of target homologs are classified as positive genomes, whereas genomes containing a single copy of the target homolog are classified as negative genomes. Target homologs that are present in multiple copies may comprise putative embedded or non-embedded target genes. Positive and negative genomes can be used to calculate several different genomic metrics (described in the following sections) that may be used to determine if the putative target genes identified in the phylogenetic tree are actual ETaGs or NETaGs.

BGC annotation: Identification and annotation of biosynthetic gene clusters comprises the identification of secondary metabolism genes, and the prediction of the group of secondary metabolism genes that constitutes a BGC. Secondary metabolism genes (or their corresponding proteins or protein domains) are genes or gene products that are not involved in primary metabolism. Examples of secondary metabolism genes include, but not limited to, the genes that encode core enzymes such as polyketide synthases (PKSs), non-ribosomal peptide synthetases (NRPSs), enzymes containing NRPS or PKS domains (e.g., PKS-like enzymes, NRPS-like enzymes, NRPS—PKS or PKS-NRPS hybrids), terpene synthases (TPs), enzymes that synthesize isoprenoids, enzymes that synthesize beta lactones, ribosomally-synthesized and post-translationally modified proteins (RIPPS), or any combination thereof, which are sometimes colocalized with tailoring enzymes. Examples of tailoring enzymes include, but are not limited to, cytochromes P450 (CYPs), methyltransferases, glycosyltransferases, etc.

BGCs can be predicted using any of a variety of software tools known to those of skill in the art. Examples include, but are not limited to, BLAST, pHMMs, the antibiotic secondary metabolite analysis shell (antiSMASH), the secondary metabolite unknown regions finder (SMURF), DeepBGC, or custom BGC prediction tools.

Correlation analysis: Clusters of Orthologous Groups (COGs) are collections of homologous genes that are useful for the study of evolutionary relationships. A COG consists of orthologs (homologous genes that have diverged in different species from a common ancestral gene) and paralogs (genes in a single species that have arisen by duplication and divergence). Scc, e.g., Tatusov, et al. (1997), “A Genomic Perspective on Protein Families”, Science 278:631-637. COGs of genes or proteins encoded thereby may be identified by performing an all-versus-all protein (amino acid) sequence search (or an all-versus-all nucleotide sequence search) of all positive and negative genomes using, for example, sequence alignment software such as BLAST, DIAMOND, or ggsearch.

In some instances, reciprocal best-hits (i.e., where the best match of a protein/gene from Genome A to Genome B is the same as the best match of a protein/gene from Genome B to Genome A) are identified and clustered into COGs using a clustering algorithm such as MCL, mmseq, usearch, CD-hit, etc. Alternatively, in some instances, unidirectional search results (rather than reciprocal search results) may be used to identify homologous proteins/genes prior to clustering.

COGs can also be identified using software tools such as OrthoMCL or OrthoFinder (or other orthogroup/pangenome identification tools), or using protein or nucleotide clustering tools such as USEARCH, CD-HIT, and MMseqs.

Co-evolution analysis: For co-evolution analysis, all genes from negative genomes are removed from consideration for all COGs. Then only COGs that have more than 3 remaining genes, each arising from a unique genome, are passed into the co-evolution analysis.

Multiple protein (amino acid) sequence alignments or DNA (nucleotide) sequence alignments are performed for all remaining COGs using, e.g., MAFFT or any other sequence alignment software. For each COG, all pairwise alignments can be trimmed based on a set of specified parameters (e.g., removal of all gaps, removal of gaps that are larger than a specified threshold (e.g., gaps of more than 30%, 20%, or 10% of the sequence in aligned sequences), keeping all gaps, etc.), followed by calculation of a percent sequence identity (e.g., the number of identical residues in the alignment). Alternatively, a sequence similarity score can be calculated based on the use of substitution matrices like BLOSUM or PAM (e.g., if protein sequences are used). The higher the percent sequence identity between two protein sequences, the more likely they are homologs and the more likely they will be assigned to the same COG. Co-evolution can be identified if the change in percent sequence identity of the proteins within one COG is correlated with the change in percent sequence identity of the proteins of another COG.

Alternatively, in some instances, phylogenetic trees can be computed from sequences (nucleotide or amino acid sequences) within each COG (e.g., by performing alignment, trimming, and phylogenetic reconstruction). Phylogenetic trees must be constrained to the topology of the species tree of genomes with genes present in both COGs being compared (e.g., after performing the step of removing genes for negative genomes from consideration and analyzing only COGs that have more than three remaining genes). Since the two COG trees are constrained to the species tree topology they will share the exact same topology. In this sense all nodes and branches are in the exact same positions, but the branch lengths (indicating the degree of divergence, and provided as an output by phylogenetic software tools such as RAxML, FastTree, IQTREE, PAULP, BEAST, etc.) may vary between the COG trees. For example, the branch length between Node_A and COG_1_genome_x may be 0.05, while the branch length between Node_A and COG_2_genome_x may be 0.075. These pairwise associations are then recorded for use in performing a correlation analysis (described below). Alternatively, in some instances, branch lengths can be the raw outputs from the phylogenetic software tool, or they can be normalized by the branch lengths of the constrained species tree, or they can be normalized through the use of a Z-score transformation or similar transformation metric. This analysis can be performed using a custom script or using tools such as the Co-Variance algorithm in PhyKIT (https://github.com/JLSteenwyk/PhyKIT).

Pairwise percent sequence identities, percent sequence similarities, or branch lengths (between pairs of genomes) for each COG are then used to calculate the degree of correlation for all pairwise COG combinations using, e.g., Pearson R, or any other correlation metric. Correlations are only computed between pairs of COGs that share at least 3 genomes.

To distinguish ETaGs or NETaGs from putative target genes and identify candidate BGCs for ETaGs or NETaGs, three different co-evolution correlation metrics may be used:
(i) Co-evolution correlation: the correlation of the pairwise percent sequence identities of COGx with the pairwise percent sequence identities of COGy.
(ii) Co-evolution rank: the rank of the correlation coefficient of the COG that contains the core synthase in ascending order in relation to the COG that contains the pETaG or pNETaG. In the case of ties for a distance score, the rank for all COGs in the tie is the lowest rank in the group.
(iii) Co-evolution slope: the orthogonal regression of the pairwise percent sequence identities of COGx with the pairwise percent sequence identities of COGy.

Co-occurrence analysis: In order to correlate the presence of a candidate BGC with additional copies of target gene homologs in a given genome with stronger statistical power, we need to limit the number of candidate BGCs by creating BGC “communities” in the selected group of genomes. One approach to doing this comprises the use of “clusteromics” (i.e., the clustering of BGCs into gene cluster families that contain orthologous BGCs) to group BGCs based on alignments of all protein sequences or nucleotide sequences in a given BGC. Alignments between all protein sequences or nucleotide sequences of a group of BGCs are performed using an alignment search tool, such as one of the programs included in the BLAST+suite or DIAMOND. Subsequently, alignments are aggregated by cluster scores describing the similarity of the BGCs. To create the cluster score, percent sequence identity of, e.g., protein sequence alignments between BGC proteins may be summed up and divided by the total number of biosynthetic proteins within a BGC, thereby creating an average percent sequence identity score for BGC to BGC comparisons. Communities of BGCs are generated by processing subsets of cluster scores for hits (i.e., BGCs that meet a threshold of at least 20%, 30%, 40%, or more than 40% average percent sequence identity) using community detection algorithms. Examples of BGC community detection algorithms include, but are not limited to, Cluster Walktrap (from https://igraph.org/) or Markov Clustering (MCL).

Alternatively, in some instances, clusteromics can be performed on a set of protein domains (or pHMMs) instead of using the full protein (or amino acid) sequences, or a phylogenetic analysis of the protein domains or protein sequences of BGCs can be used to create communities of BGCs.

Taxonomy: In some instances, one may limit the number of candidate BGCs by choosing genomes with a specific taxonomy at any level, e.g., species, genus, family, order, class, domain, etc. Genome taxonomy can be annotated based on, e.g., ribosomal RNA sequence, internal transcribed spacer (ITS) sequence, single-copy marker gene sequences, etc., by comparing them with known reference sequences.

Phylogenetic trees: In some instances, single-copy proteins or genes, or specific sequences such as that of the ITS region, can be used to create phylogenetic trees from a set of genomes. Genomes from a specific clade of the phylogenetic tree can be selected to limit the number of genomes to be used in the co-occurrence analysis.

Candidate BGC detection based on co-occurrence: To identify relevant candidate BGCs that produce a secondary metabolite having an activity against the product of the target gene, the presence of predicted BGCs is compared to the presence of single and multi-copy target gene homologs in the genomes for the selected organisms. Candidate BGCs with a hypothesized function against the target gene product should show a positive correlation with the presence of additional copies of the target gene homolog (the ETaG or NETaG clade of the phylogenetic tree), while candidate BGCs should show a negative correlation with the presence of single copies of the target gene homologs.

In some instances, a normalized distance may be used to identify the top candidate BGC hit for use in, e.g., drug development. Total positive genomes (TPG) describes the number of genomes in the ETaG or NETaG clade of the phylogenetic tree, while positive genomes (PG) describes the number of positive genomes in the BGC community. Total negative genomes (TNG) describes the number of genomes that only have a single “house-copy” of the target gene homolog, and negative genomes (NG) describes the number of negative genomes in the BGC community. The normalized distance is then given by:

$$Normalized Distance : \frac{\sqrt{{( TPG - PG )}^{2} + {( 0 - NG )}^{2}}}{\sqrt{{( TPG )}^{2} + {( TNG )}^{2}}} .$$

Co-regulation: As functionally related genes are often co-regulated, a determination of co-regulation can serve as an additional layer of information in connecting ETaGs or NETaGs to their associated BGCs. This can be achieved by identifying signatures of shared regulation, for example, the presence of shared putative cis-regulatory elements or transcription factor binding sites (TFBS) in the promoter regions of ETaGs or NETaGs and candidate BGCs. The methodology for identifying co-regulated ETaGs or NETaGs and BGCs is as follows:
1. To identify co-regulated genes, first the intergenic regions (ranging from 100 bp to 5,000 bp) of all the genes of a candidate BGCs, or COGs of candidate core synthase genes, are extracted.
2. De novo DNA motif detection is then conducted on these intergenic regions using motif detection software such as MEME (Bailey, et al. (2015) “The MEME Suite”, Nucleic Acids Res. 43 (W1): W39-49) or HOMER (Heinz, et al. (2010), “Simple Combinations of Lineage-Determining Transcription Factors Prime cis-Regulatory Elements Required for Macrophage and B Cell Identities”, Mol Cell 38 (4): 576-89).
3. Putative TFBSs, represented as position weight matrices, identified for each BGC or COG via this analysis can then be used to search the promoter regions of the target ETaGs or NETaG to evaluate whether these motifs are conserved in these regions.
4. Alternatively, de novo detected motifs from the ETaG or NETaG COG may be compared directly to motifs detected from the candidate BGC or core synthase COGs to evaluate the similarity of these motifs.
5. Detection of the BGC or core synthase motifs in the promoter region of an ETaG or NETaG, or a good match between a BGC/core synthase motif and an ETaG or NETaG motif, provides evidence of associated between the two.

Co-expression: As functionally related genes are often also co-expressed under all or a subset of conditions, an ETaG or NETaG serving as a resistance gene for a BGC would be expected to be co-expressed with BGC genes. Transcriptomics analysis can thus be used to associate an ETaG or NETaG with its cognate BGC. Data obtained from transcriptional analyses such as qPCR, microarrays, RNA-seq, NanoString, etc., conducted under multiple growth conditions (e.g., the use of different media during fermentation to induce expression of BGCs and resistance genes) or over a time-course, can be used to evaluate the correlation in expression between an ETaG or NETaG and candidate BGC genes. Candidate BGCs co-expressed with an ETaG or NETaG can be identified as follows:
1. Global transcriptomics data (e.g., RNA-seq data), obtained from multiple conditions or timepoints, are mapped to a reference genome, then read counts are computed and normalized, and differential expression analysis conducted using well established pipelines (such as Bowtie, TopHat, Cufflinks, Cuffdiff, EdgeR, or DESeq) or in-house developed pipelines.
2. Normalized read counts for each gene are then used as input for a clustering analysis, using a clustering algorithm such as K-means clustering, centroid-based clustering, density-based clustering, or hierarchical clustering, etc., to identify genes that are co-expressed with one another under all conditions analyzed.
3. Alternatively, bi-clustering approaches which cluster on the basis of both genes and conditions can be used to group genes that are co-expressed with one another under all or a subset of the conditions analyzed.
4. BGCs that are identified as being co-expressed with the ETaG or NETaG can be considered as strong candidates.

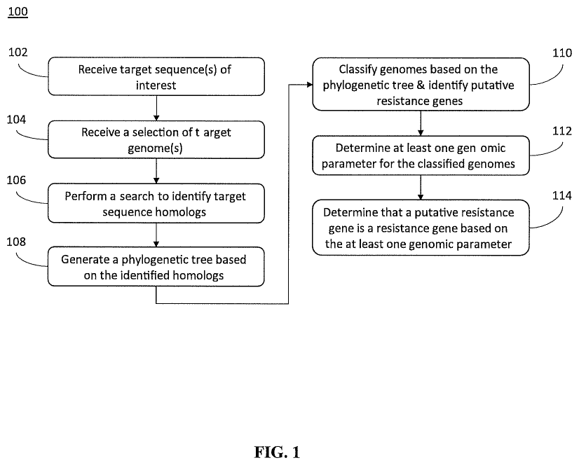

FIG. 1 provides a non-limiting example of a flowchart for a process 100 for identifying putative resistance genes (e.g., putative embedded target genes (pETaGs) and/or putative non-embedded target genes (pNETaGs)) and evaluating their likelihood of being actual resistance genes (e.g., EtaGs and/or NETaGs). Process 100 can be performed, for example, as a computer-implemented method using software running on one or more processors of one or more electronic devices, computers, or computing platforms. In some examples, process 100 is performed using a client-server system, and the blocks of process 100 are divided up in any manner between the server and a client device. In other examples, the blocks of process 100 are divided up between the server and multiple client devices. Thus, while portions of process 100 are described herein as being performed by particular devices of a client-server system, it will be appreciated that process 100 is not so limited. In other examples, process 100 is performed using only a client device or only multiple client devices. In process 100, some blocks are, optionally, combined, the order of some blocks is, optionally, changed, and some blocks are, optionally, omitted. In some examples, additional steps may be performed in combination with the process 100. Accordingly, the operations as illustrated (and described in greater detail below) are exemplary by nature and, as such, should not be viewed as limiting.

At step 102 in FIG. 1, at least one target sequence of interest is selected and/or received as input, e.g., an amino acid sequence or corresponding nucleotide sequence for a potential therapeutic target. In some instances, the selection of the at least one target sequence of interest may be provided as input by a user of a system configured to perform the computer-implemented method. In some instances, the at least one target sequence may comprise 1, 2, 3, 4, 5, 6, 7, 8, 9, 10, 100, 1000, 10,000, 20,000, 30,000, 40,000, 50,000, 60,000, 70,000, 80,000, 90,000, 100,000, or more than 100,000 target sequences (or any number of target sequences within this range).

In some instance, the at least one target sequence of interest may comprise an amino acid sequence, a nucleotide sequence, or any combination thereof. In some instances, the at least one target sequence of interest may comprise a peptide sequence or portion thereof, a protein sequence or portion thereof, a protein domain sequence or portion thereof, a gene sequence or portion thereof, or any combination thereof.

In some instances, the at least one target sequence of interest may comprise a mammalian sequence, a human sequence, an avian sequence, a reptilian sequence, an amphibian sequence, a plant sequence, a fungal sequence, a bacterial sequence, an archaea sequence, a viral sequence, or any combination thereof. For example, in some instances, the at least one target sequence of interest may comprise a mammalian target sequence, a human target sequence, an avian target sequence, a reptilian target sequence, an amphibian target sequence, a plant target sequence, a fungal target sequence, a bacterial target sequence, an archaea target sequence, a viral target sequence, or any combination thereof. In some instances, a target sequence (e.g., a human target sequence) may be a therapeutic target sequence (e.g., a human therapeutic target sequence), or a protein encoded thereby.

In some instances, the at least one target sequence of interest comprises a primary target sequence and one or more related sequences. In some instances, as noted above, the one or more related sequences may comprise sequences that are functionally-related to the primary target sequence. In some instances, the one or more related sequences may comprise sequences that are pathway-related to the primary target sequence.

At step 104 in FIG. 1, target genome(s) are selected and/or received as input, where the selection comprises a plurality of target genomes from organisms that are known to produce, or are likely to produce, secondary metabolites. In some instances, for example, the plurality of target genomes comprise plant genomes, fungal genomes, bacterial genomes, or any combination thereof.

In some instances, the selection of target genomes is provided as input by a user of a system configured to perform the computer-implemented method. The target genomes may be selected, for example, from a genomics database, e.g., a public genomics database or a proprietary genomics database. In some instances, 2, 3, 4, 5, 6, 7, 8, 9, 10, 15, 20, 25, 30, 35, 40, 45, 50, 60, 70, 80, 90, 100, 150, 200, 250, 500, 1,000, 5,000, 10,000, 20,000, 30,000, 40,000, 50,000, 60,000, 70,000, 80,000, 90,000, 100,000, or more than 100,000 target genomes (or any number of target genomes within this range) may be selected.

At step 106 in FIG. 1, a search is performed to identify homologs of the at least one target sequence in the plurality of target genomes.

In some instances, the search to identify homologs of the at least one target sequence may comprise identification of homologs based on probabilistic sequence alignment models, for example, profile hidden Markov models (pHMMs). In some instances, homologs of the at least one target sequence may be identified based on a comparison of probabilistic sequence alignment model scores to a predefined threshold. In some instances, such predetermined thresholds may be determined based on, e.g., the lowest bitscore for known homologs.

In some instances, the search to identify homologs of the at least one target sequence may comprise identification of homologs based on alignment of sequences using a local sequence alignment search tool, calculation of a sequence homology metrics based on the alignments, and comparison of the calculated sequence homology metrics to a predefined threshold. In some instances, for example, the local sequence alignment search tool may comprise BLAST, DIAMOND, HMMER, Exonerate, or ggsearch. In some instances, the predefined threshold comprises a threshold for percent sequence identity, percent sequence coverage, E-value, or bitscore value.

In some instances, the predefined threshold for percent sequence identity may be at least 20%, 30%, 40%, 50%, 60%, 70%, 80%, 85%, 90%, 95%, 96%, 97%, 98%, 99%, or higher.

In some instances, the predefined threshold for percent sequence coverage may be at least 20%, 30%, 40%, 50%, 60%, 70%, 80%, 85%, 90%, 95%, 96%, 97%, 98%, 99%, or higher.

In some instances, the predefined threshold for E-value may be at most 10, 1, 0.1, 0.001, 0.0001, 1e⁻¹⁰, 1e⁻²⁰, 1e⁻¹⁰⁰, or lower.

In some instances, the predefined threshold for bitscore may be at least 5, 10, 25, 50, 100, 250, 500, 1000, 5000, or more.

In some instances, the search to identify homologs of the at least one target sequence may comprise identification of homologs based on use of a gene and/or protein domain annotation tool. For example, in some instances the gene and/or protein domain annotation tool may comprise InterProScan or EggNOG.

At step 108 in FIG. 1, a phylogenetic tree is generated based on the identified homologs of the at least one target sequence, as described elsewhere herein.

In some instances, the generation of phylogenetic trees based on the identified homologs of the at least one target sequence may comprise one or more of: (i) alignment of the homolog sequences using an alignment software tool, (ii) trimming of the aligned homolog sequences using a sequence trimming software tool, and (iii) construction of a phylogenetic tree using phylogenetic tree building software tool. In some instances, the alignment software tool may comprise, for example, MAFFT, MUSCLE, or ClustalW. In some instances, the sequence trimming software tool may comprise, for example, trimAI, GBlocks, or ClipKIT.

In some instances, the phylogenetic tree building software tool may comprise, for example, FastTree, IQ-TREE, RAXML, MEGA, MrBayes, BEAST, or PAUP. The construction of the phylogenetic tree may be based on any of a variety of algorithms known to those of skill in the art, for example, a maximum likelihood algorithm, parsimony algorithm, neighbor joining algorithm, distance matrix algorithm, or Bayesian inference algorithm.

At step 110 in FIG. 1, the genomes of the plurality of target genomes are classified as positive genomes or negative genomes based on the phylogenetic tree (as described elsewhere herein), where positive genomes are genomes that belong to a clade for which multiple copies of the at least one target sequence homolog are present, where negative genomes are genomes that belong to a clade for which a single copy of the at least one target sequence homolog is present; and where a target sequence homolog that is present in multiple copies in a positive genome is a putative resistance gene (e.g., a putative ETaG or NETaG).

At step 112 in FIG. 1, at least one genomic parameter is determined based at least in part on the classification of positive and negative genomes, where the at least one genomic parameter is selected from the following: i) one or more scores indicative of co-occurrence of the at least one target sequence homolog (putative ETaG or NETaG) and one or more genes associated with a biosynthetic gene cluster (BGC), as described elsewhere herein; ii) one or more scores indicative of co-evolution of the at least one target sequence homolog (putative ETaG or NETaG) and one or more genes associated with a BGC, as described elsewhere herein; iii) one or more scores indicative of co-regulation of the at least one target sequence homolog (putative ETaG or NETaG) with one or more genes associated with a BGC; and iv) one or more scores indicative of co-expression of the at least one target sequence homolog (putative ETaG or NETaG) with one or more genes associated with a BGC.

In some instances, the one or more scores indicative of co-occurrence are determined based on identifying positive correlations between the presence of multiple copies of a putative ETaG or NETaG and the presence of the one or more genes of a BGC identified in positive genomes.

In some instances, identifying the positive correlations between the presence of multiple copies of the putative ETaG or NETaG and the presence of the one or more genes of a BGC identified in positive genomes may comprise the use of a clustering algorithm to cluster aligned protein sequences, aligned nucleotide sequences, aligned protein domain sequences, or aligned pHMMs for a group of BGCs to identify BGC communities within the plurality of target genomes.

In some instances, identifying the positive correlations between the presence of multiple copies of the putative ETaG or NETaG and the presence of the one or more genes of a BGC identified in positive genomes may comprise the use of a phylogenetic analysis of protein sequences or protein domains for a group of BGCs to identify BGC communities within the plurality of target genomes.

In some instances, identifying the positive correlations between the presence of multiple copies of the putative ETaG or NETaG and the presence of the one or more genes of a BGC identified in positive genomes may comprise choosing genomes with a specific taxonomy to identify BGC communities within the plurality of target genomes.

In some instances, the one or more scores indicative of co-evolution of a putative ETaG or NETaG and the one or more genes associated with a BGC may be determined based on a co-evolution correlation score, a co-evolution rank score, a co-evolution slope score, or any combination thereof.

In some instances, the co-evolution correlation score (or co-evolution correlation coefficient) may be based on a correlation between pairwise percent sequence identities of a cluster of orthologous groups (COG) for the putative ETaG or NETaG and pairwise percent sequence identities of a cluster of orthologous groups (COG) for one of the one or more genes associated with a BGC (as described elsewhere herein). In some instances, the co-evolution correlation score (or co-evolution correlation coefficient) may range in value from −1.0 to 1.0. In some instances, the co-evolution correlation score (or co-evolution correlation coefficient) may have a value of −1.0, −0.8, −0.6, −0.4, −0.2, 0, 0.2, 0.4, 0.6, 0.8, 1.0, or any value within this range.

In some instances, the co-evolution rank score (or co-evolution rank) may be based on a ranking of the correlation coefficient of a COG that contains one of the one or more genes associated with a BGC in ascending order in relation to a COG that contains the putative ETaG or NETaG (as described elsewhere herein). In some instances, the co-evolution rank may range in value from 1 to 10,000. In some instances, the co-evolution rank may have a value of 1, 10, 20, 40, 60, 80, 100, 200, 400, 600, 800, 1000, 2000, 4000, 6000, 8000, or 10,000, or any value within this range. In the case of ties for a distance score, the rank for all COGs in the tie may be set equal to a lowest rank in the group.

In some instances, the co-evolution slope score may be based on an orthogonal regression of pairwise percent sequence identities of a COG for the putative ETaG or NETaG and pairwise percent sequence identities of a COG for one of the one or more genes associated with a BGC (as described elsewhere herein). In some instances, the co-evolution slope score may range in value from about 0.75 to about 1.25. In some instances, the co-evolution slope score may have a value of at least 0.75, at least 0.8, at least 0.85, at least 0.9, at least 0.95, at least 1.0, at least 1.05, at least 1.1, at least 1.15, at least 1.20, or at least 1.25. In some instances, the co-evolution slope score may have a value of at most 1.25, at most 1.20, at most 1.15, at most 1.10, at most 1.10, at most 1.05, at most 1.0, at most 0.95, at most 0.90, at most 0.85, at most 0.80, or at most 0.75. Any of the lower and upper values described in this paragraph may be combined to form a range included within the present disclosure, for example, in some instances the co-evolution slope score may range from about 0.80 to about 1.1. Those of skill in the art will recognize that the co-evolution slope score may have any value within this range, e.g., about 0.98.

In some instances, only COGs arising from unique positive genomes that have more than three genes remaining after removing corresponding genes from negative genomes are used to evaluate a co-evolution correlation score, a co-evolution rank score, or a co-evolution slope score.

In some instances, the one or more scores indicative of co-regulation may be based on, for example, the detection of DNA motifs from intergenic sequences of the one or more genes associated with a BGC and the putative resistance gene, as described elsewhere herein.

In some instances, the one or more scores indicative of co-expression may be based on, for example, a differential expression analysis and/or a clustering analysis of global transcriptomics data, as described elsewhere herein.

In some instances, the one or more genes associated with a biosynthetic gene cluster (BGC) may comprise, for example, an anchor gene, a core synthase gene, a biosynthetic gene, a gene not involved in the biosynthesis of a secondary metabolite produced by the BGC, or any combination thereof.

At step 114 in FIG. 1, a likelihood that the putative resistance gene (e.g., a pETaG or pNETaG) is an actual resistance gene (e.g., an ETaG or NETaG) is determined based on the at least one genomic parameter determined in step 112. In some instances, the likelihood that the putative resistance gene is an actual resistance gene may be output and/or reported as a probability, e.g., a 10%, 20%, 30%, 40%, 50%, 60%, 70%, 80%, 90%, 95%, 98%, or 99% probability that the putative resistance gene is an actual resistance gene. In some instances, the likelihood that the putative resistance gene is an actual resistance gene may be output and/or reported as a probability having any value within this range.

In some instances, determining the likelihood that the putative resistance gene (e.g., pETaG or pNETaG) is an actual resistance gene (e.g., ETaG or NETaG) may comprise outputting or reporting a binary classification (e.g., a yes/no answer) of the likelihood based on comparing the at least one determined genomic parameter to at least one predetermined threshold. In some instances, for example the at least one predetermined threshold may comprise a predetermined threshold for a co-occurrence score, a co-evolution correlation score, a co-regulation score, and/or a co-expression score.

In some instances, for example, a predetermined threshold for co-occurrence score may comprise inclusion of the top 20, top 15, top 10, or top 5 co-occurring BGCs as ranked by normalized distance. The co-occurrence rank may be used to confirm an association between BGCs and their putative resistance genes (e.g., pETaGs or pNETaGs). A normalized distance may be calculated from the occurrence of BGC genes and resistance genes (e.g., ETaGs or NETaGs) throughout positive and negative genomes. BGC genes may be ranked by their normalized distance (calculated from positive and negative genome counts).

In some instances, a predetermined threshold for co-evolution correlation score may comprise a co-evolution correlation coefficient of greater than or equal to 0.6, 0.7, 0.8, 0.9, 0.95, or greater. In some instances, a predetermined threshold for co-evolution correlation score may have any value within this range.

In some instances, a predetermined threshold for co-evolution rank score (or co-evolution rank) may comprise a rank of less than 5, less than 10, less than 20, less than 40, less than 60, less than 80, less than 100, less than 200, less than 400, less than 600, less than 800, less than 1000, less than 2000, less than 4000, less than 6000, less than 8000, or less than 10,000. In some instances, a predetermined threshold for co-evolution rank score (or co-evolution rank) may comprise a rank of any value within this range of values.

In some instances, a predetermined threshold for co-evolution slope may comprise a co-evolution slope value of between about 0.75 and about 1.25. In some instances, the predetermined threshold for co-evolution slope score may have a value of at least 0.75, at least 0.8, at least 0.85, at least 0.9, at least 0.95, at least 1.0, at least 1.05, at least 1.1, at least 1.15, at least 1.20, or at least 1.25. In some instances, the predetermined threshold for co-evolution slope score may have a value of at most 1.25, at most 1.20, at most 1.15, at most 1.10, at most 1.10, at most 1.05, at most 1.0, at most 0.95, at most 0.90, at most 0.85, at most 0.80, or at most 0.75. Any of the lower and upper values described in this paragraph may be combined to form a range included within the present disclosure, for example, in some instances the predetermined threshold for co-evolution slope score may range from about 0.80 to about 1.1. In some instances, the predetermined threshold for co-evolution slope score may have any value within this range, e.g., about 1.07.

In some instances, a predetermined threshold for a co-regulation score may comprise detecting a DNA motif in the upstream intergenic sequence of one or more members of the BGC members and the putative resistance gene with a p-value of less than or equal to 0.1, 0.09, 0.08, 0.07, 0.06, or 0.05.

In some instances, a predetermined threshold for a co-expression score may be based on the values determined for a differential expression analysis metric such as a Spearman correlation coefficient, Kolmogorov-Smirnov distance, Euclidean distance, Kullback-Leibler divergence, or adjacency difference (see, e.g., Gonzalez-Valbuena, et al. (2017), “Metrics to Estimate Differential Co-Expression Networks”, BioData Mining 10:32). In these instances, a predetermined threshold for co-expression may comprise a co-expression score of greater than or equal to 0.6, 0.7, 0.8, 0.9, 0.95, or greater. In some instances, a predetermined threshold for co-expression score may have any value within this range.

In some instances, e.g., where co-expression scores are based on a clustering analysis of global transcriptomics data, a predetermined threshold for a co-expression score may not be used.

Methods for Predicting the Function of a Secondary Metabolite and/or Identifying a Secondary Metabolite Having an Activity of Interest

Also disclosed herein are computer-implemented methods for predicting a function of a secondary metabolite and/or for identifying a biosynthetic gene cluster (BGC) that encodes biosynthetic enzymes for producing a secondary metabolite having an activity of interest.

For example, in some instances a computer-implemented method for predicting a function of a secondary metabolite may comprise: receiving a selection of at least one target sequence of interest, wherein the at least one target sequence of interest corresponds to a gene sequence associated with a biosynthetic gene cluster (BGC) known to produce the secondary metabolite; receiving a selection of target genomes from a genomics database, wherein the selection of target genomes comprises a plurality of target genomes from organisms that are known to produce secondary metabolites; performing a search to identify homologs of the at least one target sequence in the plurality of target genomes; generating a phylogenetic tree based on the identified homologs of the at least one target sequence; classifying the genomes of the plurality of target genomes as positive genomes or negative genomes based on the phylogenetic tree, wherein positive genomes are genomes that belong to a clade for which multiple copies of the at least one target sequence homolog are present, wherein negative genomes are genomes that belong to a clade for which a single copy of the at least one target sequence homolog is present; and wherein a target sequence homolog that is present in multiple copies in a positive genome is a putative resistance gene; determining, based at least in part on the classification of positive and negative genomes, at least one genomic parameter selected from the following: i) one or more scores indicative of co-occurrence of the at least one target sequence homolog (putative resistance gene) and one or more genes associated with the BGC; ii) one or more scores indicative of co-evolution of the at least one target sequence homolog (putative resistance gene) and one or more genes associated with the BGC; iii) one or more scores indicative of co-regulation of the at least one target sequence homolog (putative resistance gene) with one or more genes associated with the BGC; and iv) one or more scores indicative of co-expression of the at least one target sequence homolog (putative resistance gene) with one or more genes associated with the BGC; and determining, based on the at least one genomic parameter, a likelihood that the putative resistance gene is a resistance gene that encodes a protein target that is acted upon by the secondary metabolite.

In some instances, the selection of at least one target sequence of interest may be provided as input by a user of a system configured to perform the computer-implemented method.

In some instances, as described elsewhere herein, the at least one target sequence of interest may comprise an amino acid sequence, a nucleotide sequence, or any combination thereof. In some instances, the at least one target sequence of interest comprises a peptide sequence or portion thereof, a protein sequence or portion thereof, a protein domain sequence or portion thereof, a gene sequence or portion thereof, or any combination thereof.

In some instances, the selection of target genomes may be provided as input by a user of a system configured to perform the computer-implemented method.

In some instances, the plurality of target genomes may comprise plant genomes, fungal genomes, bacterial genomes, or any combination thereof. In some instances, the genomics database comprises a public genomics database or a proprietary genomics database.

In some instances, the search to identify homologs of the at least one target sequence may comprise identification of homologs based on probabilistic sequence alignment models. In some instances, the probabilistic sequence alignment models are profile hidden Markov models (pHMMs). In some instances, homologs are identified based on a comparison of probabilistic sequence alignment model scores to a predefined threshold as described elsewhere herein. In some instances, for example, such predetermined thresholds may be determined based on the lowest bitscore for known homologs.

In some instances, the search to identify homologs of the at least one target sequence may comprise identification of homologs based on alignment of sequences using a local sequence alignment search tool, calculation of a sequence homology metrics based on the alignments, and comparison of the calculated sequence homology metrics to a predefined threshold. In some instances, as described elsewhere herein, the predefined threshold comprises a threshold for percent sequence identity, percent sequence coverage, E-value, or bitscore value.

In some instances, the predefined threshold for percent sequence identity may be at least 20%, 30%, 40%, 50%, 60%, 70%, 80%, 85%, 90%, 95%, 96%, 97%, 98%, 99%, or higher.

In some instances, the predefined threshold for percent sequence coverage may be at least 20%, 30%, 40%, 50%, 60%, 70%, 80%, 85%, 90%, 95%, 96%, 97%, 98%, 99%, or higher.

In some instances, the predefined threshold for E-value may be at most 10, 1, 0.1, 0.001, 0.0001, 1e⁻¹⁰, 1e⁻²⁰, 1e⁻¹⁰⁰, or lower.

In some instances, the predefined threshold for bitscore may be at least 5, 10, 25, 50, 100, 250, 500, 1000, 5000, or more.

In some instances, the search to identify homologs of the at least one target sequence comprises identification of homologs based on use of a gene and/or protein domain annotation tool.

In some instances, the generation of phylogenetic trees based on the identified homologs of the at least one target sequence may comprise alignment of homolog sequences using an alignment software tool, trimming of the aligned homolog sequences using a sequence trimming software tool, and construction of a phylogenetic tree using phylogenetic tree building software tool, as described elsewhere herein.

In some instances, the at least one target sequence of interest may comprise, for example, a known ETaG sequence, a known NETaG sequence, or a known core synthase gene sequence.

In some instances, determining the likelihood that the putative resistance gene is a resistance gene that encodes a protein target that is acted upon by the secondary metabolite may comprise comparing the at least one determined genomic parameter to at least one predetermined threshold. In some instances, for example, the at least one predetermined threshold may comprise a predetermined threshold for a co-occurrence score, a co-evolution score, a co-regulation score, and/or a co-expression score. Examples of such predetermined thresholds are described elsewhere herein.

As another non-limiting example, computer-implemented methods for identifying a biosynthetic gene cluster (BGC) that encodes biosynthetic enzymes for producing a secondary metabolite having an activity of interest are also disclosed, the methods comprising: receiving a selection of at least one target sequence of interest, wherein the at least one target sequence of interest comprises a sequence that encodes a therapeutic target of interest; receiving a selection of target genomes from a genomics database, wherein the selection of target genomes comprises a plurality of target genomes from organisms that are known to produce secondary metabolites; performing a search to identify homologs of the at least one target sequence in the plurality of target genomes; generating a phylogenetic tree based on the identified homologs of the at least one target sequence; classifying the genomes of the plurality of target genomes as positive genomes or negative genomes based on the phylogenetic tree, wherein positive genomes are genomes that belong to a clade for which multiple copies of the at least one target sequence homolog are present, wherein negative genomes are genomes that belong to a clade for which a single copy of the at least one target sequence homolog is present; and wherein a target sequence homolog that is present in multiple copies in a positive genome is a putative resistance gene; determining, based at least in part on the classification of positive and negative genomes, at least one genomic parameter selected from the following: i) one or more scores indicative of co-occurrence of the at least one target sequence homolog (putative resistance gene) and one or more genes associated with a biosynthetic gene cluster (BGC); ii) one or more scores indicative of co-evolution of the at least one target sequence homolog (putative resistance) and one or more genes associated with a BGC; iii) one or more scores indicative of co-regulation of the at least one target sequence homolog (putative resistance gene) with one or more genes associated with a BGC; and iv) one or more scores indicative of co-expression of the at least one target sequence homolog (putative resistance gene) with one or more genes associated with a BGC; and determining, based on the at least one genomic parameter, a likelihood that the putative resistance gene is an actual resistance gene associated with a BGC that produces a secondary metabolite that acts upon a protein product encoded by the resistance gene.

In some instances, the selection of at least one target sequence of interest may be provided as input by a user of a system configured to perform the computer-implemented method. In some instances, as described elsewhere herein, the at least one target sequence of interest comprises an amino acid sequence, a nucleotide sequence, or any combination thereof. In some instances, the at least one target sequence of interest comprises a peptide sequence or portion thereof, a protein sequence or portion thereof, a protein domain sequence or portion thereof, a gene sequence or portion thereof, or any combination thereof.

In some instances, the selection of target genomes may be provided as input by a user of a system configured to perform the computer-implemented method. In some instances, as described elsewhere herein, the plurality of target genomes may comprise plant genomes, fungal genomes, bacterial genomes, or any combination thereof. In some instances, the genomics database may comprise a public genomics database or a proprietary genomics database.

In some instances, the search to identify homologs of the at least one target sequence may comprise identification of homologs based on probabilistic sequence alignment models. In some instances, the probabilistic sequence alignment models are profile hidden Markov models (pHMMs). In some instances, homologs are identified based on a comparison of probabilistic sequence alignment model scores to a predefined threshold. In some instances, such predetermined thresholds may be determined based on, e.g., the lowest bitscore for known homologs.

In some instances, the search to identify homologs of the at least one target sequence may comprise identification of homologs based on alignment of sequences using a local sequence alignment search tool, calculation of a sequence homology metrics based on the alignments, and comparison of the calculated sequence homology metrics to a predefined threshold. In some instances, the predefined threshold comprises a threshold for percent sequence identity, percent sequence coverage, E-value, or bitscore value.

In some instances, the predefined threshold for percent sequence identity may be at least 20%, 30%, 40%, 50%, 60%, 70%, 80%, 85%, 90%, 95%, 96%, 97%, 98%, 99%, or higher.

In some instances, the predefined threshold for percent sequence coverage may be at least 20%, 30%, 40%, 50%, 60%, 70%, 80%, 85%, 90%, 95%, 96%, 97%, 98%, 99%, or higher.

In some instances, the predefined threshold for E-value may be at most 10, 1, 0.1, 0.001, 0.0001, 1e⁻¹⁰, 1e⁻²⁰, 1e⁻¹⁰⁰, or lower.

In some instances, the predefined threshold for bitscore may be at least 5, 10, 25, 50, 100, 250, 500, 1000, 5000, or more.

In some instances, the search to identify homologs of the at least one target sequence may comprise identification of homologs based on use of a gene and/or protein domain annotation tool.

In some instances, the generation of phylogenetic trees based on the identified homologs of the at least one target sequence may comprise alignment of homolog sequences using an alignment software tool, trimming of the aligned homolog sequences using a sequence trimming software tool, and construction of a phylogenetic tree using phylogenetic tree building software tool, as described elsewhere herein.

In some instances, determining the likelihood that the putative resistance gene is an actual resistance gene associated with the BGC that produces the secondary metabolite may comprise comparing the at least one determined genomic parameter to at least one predetermined threshold. In some instances, for example, the at least one predetermined threshold may comprise a predetermined threshold for a co-occurrence score, a co-evolution score, a co-regulation score, and/or a co-expression score. Examples of such predetermined thresholds have been described elsewhere herein.

In some instances, the computer-implemented methods described herein may further comprise performing an in vitro assay to test a secondary metabolite produced by the identified BGC for activity against the therapeutic target of interest, as described elsewhere herein.

In some instances, the computer-implemented methods described herein may further comprise performing an in vivo assay to test a secondary metabolite produced by the identified BGC for activity against the therapeutic target of interest, as described elsewhere herein.

### Applications

The computer-based methods described herein have various applications including, for example, identification of homologs or orthologs of one or more target sequences (e.g., gene sequences) of interest in one or more target genomes, identification of a resistance gene against a secondary metabolite produced by a BGC in a target genome, predicting a function of a secondary metabolite produced by a BGC, and/or identifying a BGC that encodes biosynthetic enzymes for producing a secondary metabolite having an activity of interest (e.g., a therapeutic activity of interest), etc.

In some instances, the present disclosure provides methods (e.g., computer-implemented methods) for identifying embedded target genes (ETaGs) and/or non-embedded target genes (NETaGs) that may comprise: receiving a selection of at least one target sequence of interest; receiving a selection of target genomes from a genomics database, wherein the selection of target genomes comprises a plurality of target genomes from organisms that are known to produce, or are likely to produce, secondary metabolites; performing a search to identify homologs of the at least one target sequence in the plurality of target genomes; generating a phylogenetic tree based on the identified homologs of the at least one target sequence; classifying the genomes of the plurality of target genomes as positive genomes or negative genomes based on the phylogenetic tree, wherein positive genomes are genomes that belong to a clade for which multiple copies of the at least one target sequence homolog are present, wherein negative genomes are genomes that belong to a clade for which a single copy of the at least one target sequence homolog is present; and wherein a target sequence homolog that is present in multiple copies in a positive genome is a putative ETaG or NETaG; determining, based at least in part on the classification of positive and negative genomes, at least one genomic parameter selected from the following: i) one or more scores indicative of co-occurrence of the at least one target sequence homolog (putative ETaG or NETaG) and one or more genes associated with a biosynthetic gene cluster (BGC); ii) one or more scores indicative of co-evolution of the at least one target sequence homolog (putative ETaG or NETaG) and one or more genes associated with a BGC; iii) one or more scores indicative of co-regulation of the at least one target sequence homolog (putative ETaG or NETaG) with one or more genes associated with a BGC; and iv) one or more scores indicative of co-expression of the at least one target sequence homolog (putative ETaG or NETaG) with one or more genes associated with a BGC; and determining, based on the at least one genomic parameter, a likelihood that the putative ETaG or NETaG is an embedded target gene (ETaG) or non-embedded target gene (NETaG).

In some instances, the present disclosure provides methods (e.g., computer-implemented methods) for predicting a function of a secondary metabolite that may comprise: receiving a selection of at least one target sequence of interest, wherein the at least one target sequence of interest corresponds to a gene sequence associated with a biosynthetic gene cluster (BGC) known to produce the secondary metabolite; receiving a selection of target genomes from a genomics database, wherein the selection of target genomes comprises a plurality of target genomes from organisms that are known to produce secondary metabolites; performing a search to identify homologs of the at least one target sequence in the plurality of target genomes; generating a phylogenetic tree based on the identified homologs of the at least one target sequence; classifying the genomes of the plurality of target genomes as positive genomes or negative genomes based on the phylogenetic tree, wherein positive genomes are genomes that belong to a clade for which multiple copies of the at least one target sequence homolog are present, wherein negative genomes are genomes that belong to a clade for which a single copy of the at least one target sequence homolog is present; and wherein a target sequence homolog that is present in multiple copies in a positive genome is a putative resistance gene; determining, based at least in part on the classification of positive and negative genomes, at least one genomic parameter selected from the following: i) one or more scores indicative of co-occurrence of the at least one target sequence homolog (putative resistance gene) and one or more genes associated with the BGC; ii) one or more scores indicative of co-evolution of the at least one target sequence homolog (putative resistance gene) and one or more genes associated with the BGC; iii) one or more scores indicative of co-regulation of the at least one target sequence homolog (putative resistance gene) with one or more genes associated with the BGC; and iv) one or more scores indicative of co-expression of the at least one target sequence homolog (putative resistance gene) with one or more genes associated with the BGC; and determining, based on the at least one genomic parameter, a likelihood that the putative resistance gene is a resistance gene that encodes a protein target that is acted upon by the secondary metabolite.

In some instances, the present disclosure provides methods (e.g., computer-implemented methods) for identifying a biosynthetic gene cluster (BGC) that encodes biosynthetic enzymes for producing a secondary metabolite having an activity of interest that may comprise: receiving a selection of at least one target sequence of interest, wherein the at least one target sequence of interest comprises a sequence that encodes a therapeutic target of interest; receiving a selection of target genomes from a genomics database, wherein the selection of target genomes comprises a plurality of target genomes from organisms that are known to produce secondary metabolites; performing a search to identify homologs of the at least one target sequence in the plurality of target genomes; generating a phylogenetic tree based on the identified homologs of the at least one target sequence; classifying the genomes of the plurality of target genomes as positive genomes or negative genomes based on the phylogenetic tree, wherein positive genomes are genomes that belong to a clade for which multiple copies of the at least one target sequence homolog are present, wherein negative genomes are genomes that belong to a clade for which a single copy of the at least one target sequence homolog is present; and wherein a target sequence homolog that is present in multiple copies in a positive genome is a putative resistance gene; determining, based at least in part on the classification of positive and negative genomes, at least one genomic parameter selected from the following: i) one or more scores indicative of co-occurrence of the at least one target sequence homolog (putative resistance gene) and one or more genes associated with a biosynthetic gene cluster (BGC); ii) one or more scores indicative of co-evolution of the at least one target sequence homolog (putative resistance) and one or more genes associated with a BGC; iii) one or more scores indicative of co-regulation of the at least one target sequence homolog (putative resistance gene) with one or more genes associated with a BGC; and iv) one or more scores indicative of co-expression of the at least one target sequence homolog (putative resistance gene) with one or more genes associated with a BGC; and determining, based on the at least one genomic parameter, a likelihood that the putative resistance gene is an actual resistance gene associated with a BGC that produces a secondary metabolite that acts upon a protein product encoded by the resistance gene.

In some instances, the methods (e.g., computer-implemented methods) of the present disclosure may further comprise performing an in vitro assay, for example, an assay to detect or measure an activity (e.g., a receptor binding activity, an enzyme activation activity, an enzyme inhibition activity, etc.) of a secondary metabolite (or analog thereof) on a mammalian (e.g., human) protein encoded by a mammalian (e.g., human) gene that is homologous to an ETaG or NETag identified in an organism comprising a biosynthetic gene cluster (BGC) that produces the secondary metabolite. In some instances, the methods may further comprise performing an in vitro assay to detect or measure an activity (e.g., a receptor binding activity, an enzyme activation activity, an enzyme inhibition activity, etc.) of a secondary metabolite (or analog thereof) on a protein (e.g., a reptilian, avian, amphibian, plant, fungal, bacterial, or viral protein) encoded by a reptilian, avian, amphibian, plant, fungal, bacterial, or viral gene that is homologous to an ETaG or NETag identified in an organism comprising a biosynthetic gene cluster (BGC) that produces the secondary metabolite.

In some instances, the methods (e.g., computer-implemented methods) of the present disclosure may further comprise performing an in vivo assay, for example, an assay to detect or measure an activity (e.g., a receptor binding activity, an enzyme activation activity, an enzyme inhibition activity, an intracellular signaling pathway activity, a disease response, etc.) of a secondary metabolite (or analog thereof) on a mammalian (e.g., human) protein encoded by a mammalian (e.g., human) gene that is homologous to an ETaG or NETag identified in an organism comprising a biosynthetic gene cluster (BGC) that produces the secondary metabolite. In some instances, the methods may further comprise performing an in vivo assay to detect or measure an activity (e.g., a receptor binding activity, an enzyme activation activity, an enzyme inhibition activity, an intracellular signaling pathway activity, a disease response, etc.) of a secondary metabolite (or analog thereof) on a protein (e.g., a reptilian, avian, amphibian, plant, fungal, bacterial, or viral protein) encoded by a reptilian, avian, amphibian, plant, fungal, bacterial, or viral gene that is homologous to an ETaG or NETag identified in an organism comprising a biosynthetic gene cluster (BGC) that produces the secondary metabolite.

In some instances, the methods of the present disclosure may be used, for example, for identifying and/or characterizing a mammalian (e.g., human) target of a secondary metabolite (or analog thereof) produced by a BGC. In some instances, the methods of the present disclosure may be used for identifying and/or characterizing a reptilian, avian, amphibian, plant, fungal, bacterial, viral target of a secondary metabolite (or analog thereof) produced by a BGC, or a target from any other organism.

In some instances, the methods of the present disclosure may be used, for example, for drug discovery activities, e.g., to identify small molecule modulators of a mammalian (e.g., human) target gene. In some instances, the methods of the present disclosure may be used to identify small molecule modulators of a reptilian target gene, an avian target gene, an amphibian target gene, a plant target gene, a fungal target gene, a bacterial target gene, a viral target gene, or a target gene from any other organism.

In some instances, the secondary metabolite is a product of enzymes encoded by the BGC or a salt thereof, including an unnatural salt. In some instances, the secondary metabolite or analog thereof is an analog of a product of enzymes encoded by the BGC, e.g., a small molecule compound having the same core structure as the secondary metabolite, or a salt thereof.

In some instances, the present disclosure provides methods for modulating a human target (or a target from another organism), comprising: providing a secondary metabolite produced by enzymes encoded by a BGC, or an analog thereof, wherein the human target (or a nucleic acid sequence encoding the human target) is homologous to an ETaG or NETaG that is associated with the BGC as determined using any one of the methods described herein.

In some instances, the present disclosure provides methods for treating a condition, disorder, or disease associated with a human target (or a target from another organism), comprising administering to a subject susceptible to, or suffering therefrom, a secondary metabolite produced by enzymes encoded by a BGC, or an analog thereof, wherein the human target (or a nucleic acid sequence encoding the human target) is homologous to an ETaG or NETaG that is associated with the BGC as determined using any one of the methods described herein.

In some instances, the secondary metabolite is produced by a fungus. In some instances, the secondary metabolite is acyclic. In some instances, the secondary metabolite is a polyketide. In some instances, the secondary metabolite is a terpene compound. In some instances, the secondary metabolite is a non-ribosomally synthesized peptide.

In some instances, an analog of a substance (e.g., secondary metabolite) that shares one or more particular structural features, elements, components, or moieties with a reference substance. Typically, an analog shows significant structural similarity with the reference substance, for example sharing a core or consensus structure, but also differs in certain discrete ways. In some instances, an analog is a substance that can be generated from the reference substance, e.g., by chemical manipulation of the reference substance. In some instances, an analog is a substance that can be generated through performance of a synthetic process substantially similar to (e.g., sharing a plurality of steps with) one that generates the reference substance. In some instances, an analog is or can be generated through performance of a synthetic process different from that used to generate the reference substance. In some instances, an analog of a substance is the substance being substituted at one or more of its substitutable positions.

In some instances, an analog of a product comprises the structural core of a product. In some instances, a biosynthetic product is cyclic, e.g., monocyclic, bicyclic, or polycyclic, and the structural core of the product is or comprises the monocyclic, bicyclic, or polycyclic ring system. In some instances, the structural core of the product comprises one ring of the bicyclic or polycyclic ring system of the product. In some instances, a product is or comprises a polypeptide, and a structural core is the backbone of the polypeptide. In some instances, a product is or comprises a polyketide, and a structural core is the backbone of the polyketide. In some instances, an analog is a substituted biosynthetic product comprising one or more suitable substituents.

### Systems

Also disclosed herein are systems designed to implement any of the disclosed methods for identifying resistance genes (e.g., ETaGs or NETaGs). The systems may comprise, for example, one or more processors, and a memory unit communicatively coupled to the one or more processors and configured to store instructions that, when executed by the one or more processors, cause the system to: receive a selection of at least one target sequence of interest; receive a selection of target genomes from a genomics database, wherein the selection of target genomes comprises a plurality of target genomes from organisms that are known to produce, or are likely to produce, secondary metabolites; perform a search to identify homologs of the at least one target sequence in the plurality of target genomes; generate a phylogenetic tree based on the identified homologs of the at least one target sequence; classify the genomes of the plurality of target genomes as positive genomes or negative genomes based on the phylogenetic tree, wherein positive genomes are genomes that belong to a clade for which multiple copies of the at least one target sequence homolog are present, wherein negative genomes are genomes that belong to a clade for which a single copy of the at least one target sequence homolog is present; and wherein a target sequence homolog that is present in multiple copies in a positive genome is a putative resistance gene (e.g., a putative ETaG or NETaG); determine, based at least in part on the classification of positive and negative genomes, at least one genomic parameter selected from the following: i) one or more scores indicative of co-occurrence of the at least one target sequence homolog (putative resistance gene) and one or more genes associated with a biosynthetic gene cluster (BGC); ii) one or more scores indicative of co-evolution of the at least one target sequence homolog (putative resistance gene) and one or more genes associated with a BGC; iii) one or more scores indicative of co-regulation of the at least one target sequence homolog (putative resistance gene) with one or more genes associated with a BGC; and iv) one or more scores indicative of co-expression of the at least one target sequence homolog (putative resistance gene) with one or more genes associated with a BGC; and determine, based on the at least one genomic parameter, a likelihood that the putative resistance gene (e.g., pETaG or pNETaG) is an actual resistance gene (e.g., an embedded target gene (ETaG) or non-embedded target gene (NETaG)). In some instances, determining the likelihood that the putative resistance gene (e.g., pETaG or pNETaG) is a resistance gene (e.g., ETaG or NETaG) comprises comparing the at least one determined genomic parameter to at least one predetermined threshold. Examples of such predetermined thresholds are described elsewhere herein.

### Computing Devices and Systems

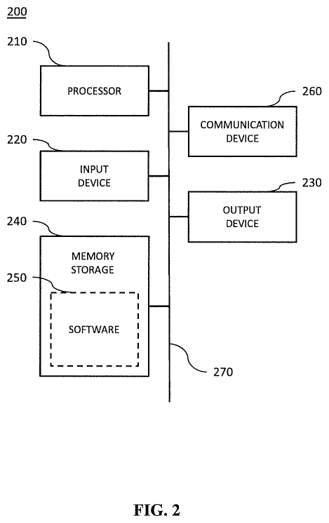

FIG. 2 illustrates an example of a computing device in accordance with one or more examples of the disclosure. Device 200 can be a host computer connected to a network. Device 200 can be a client computer or a server. As shown in FIG. 2, device 200 can be any suitable type of microprocessor-based device, such as a personal computer, workstation, server, or handheld computing device (portable electronic device), such as a phone or tablet. The device can include, for example, one or more of processor 210, input device 220, output device 230, storage 240, and communication device 260. Input device 220 and output device 230 can generally correspond to those described above, and they can either be connectable or integrated with the computer.

Input device 220 can be any suitable device that provides input, such as a touch screen, keyboard or keypad, mouse, or voice-recognition device. Output device 230 can be any suitable device that provides output, such as a touch screen, haptics device, or speaker.

Storage 240 can be any suitable device that provides storage, such as an electrical, magnetic, or optical memory including a RAM, cache, hard drive, or removable storage disk. Communication device 260 can include any suitable device capable of transmitting and receiving signals over a network, such as a network interface chip or device. The components of the computer can be connected in any suitable manner, such as via a physical bus 270 or wirelessly.

Software 250, which can be stored in memory/storage 240 and executed by processor 210, can include, for example, the programming that embodies the functionality of the present disclosure (e.g., as embodied in the devices described above).

Software 250 can also be stored and/or transported within any non-transitory computer-readable storage medium for use by or in connection with an instruction execution system, apparatus, or device, such as those described above, that can fetch instructions associated with the software from the instruction execution system, apparatus, or device and execute the instructions. In the context of this disclosure, a computer-readable storage medium can be any medium, such as storage 240, that can contain or store programming for use by or in connection with an instruction execution system, apparatus, or device.

Software 250 can also be propagated within any transport medium for use by or in connection with an instruction execution system, apparatus, or device, such as those described above, that can fetch instructions associated with the software from the instruction execution system, apparatus, or device and execute the instructions. In the context of this disclosure, a transport medium can be any medium that can communicate, propagate, or transport programming for use by or in connection with an instruction execution system, apparatus, or device. The transport readable medium can include, but is not limited to, an electronic, magnetic, optical, electromagnetic, or infrared wired or wireless propagation medium.

Device 200 may be connected to a network, which can be any suitable type of interconnected communication system. The network can implement any suitable communications protocol and can be secured by any suitable security protocol. The network can comprise network links of any suitable arrangement that can implement the transmission and reception of network signals, such as wireless network connections, Tl or T3 lines, cable networks, DSL, or telephone lines.

Device 200 can implement any operating system suitable for operating on the network. Software 250 can be written in any suitable programming language, such as C, C++, Java, or Python. In various embodiments, application software embodying the functionality of the present disclosure can be deployed in different configurations, such as in a client/server arrangement or through a web browser as a web-based application or web service, for example.

### EXAMPLES

### Example 1-Using NETaGs to Identify a BGC with a Specific Target that May have Therapeutic Applications

Succinate dehydrogenase complex subunit C (SDHC) inhibitor: a collection of protein sequences from a diverse set of fungal genomes from different taxa was annotated using InterProScan and searched for proteins annotated with Interpro ID IPR000701 (Succinate dehydrogenase/fumarate reductase type B, transmembrane subunit) to identify succinate dehydrogenase complex subunit C (SDHC) homologs in the set of genomes. Genomes with single copies of a Interpro ID IPR000701 homolog were designated negative genomes while genomes with multiple copies of a Interpro ID IPR000701 homolog were designated positive genomes. NETaGs are the copies of Succinate dehydrogenase complex subunit C (SDHC) that confer resistance to the product of the gene cluster. All SDHC protein sequences were aligned using MAFFT and trimmed using trimAI to remove gaps. The resulting trimmed multiple sequence alignment was processed with IQ-TREE to create a maximum likelihood phylogeny of SDHC homologs. NETaGs can be identified by their location within the phylogenetic tree. NETaGs from several fungal genera will cluster together in one branch or several close branches of the phylogenetic tree, while the housekeeping copies show larger phylogenetic distances and only exhibit proteins from a single fungal genus in their branch. Furthermore, the NETaG clade only includes proteins from multi-copy genomes, while the housekeeping copies exhibit proteins together in clades from single and multi-copy genomes.

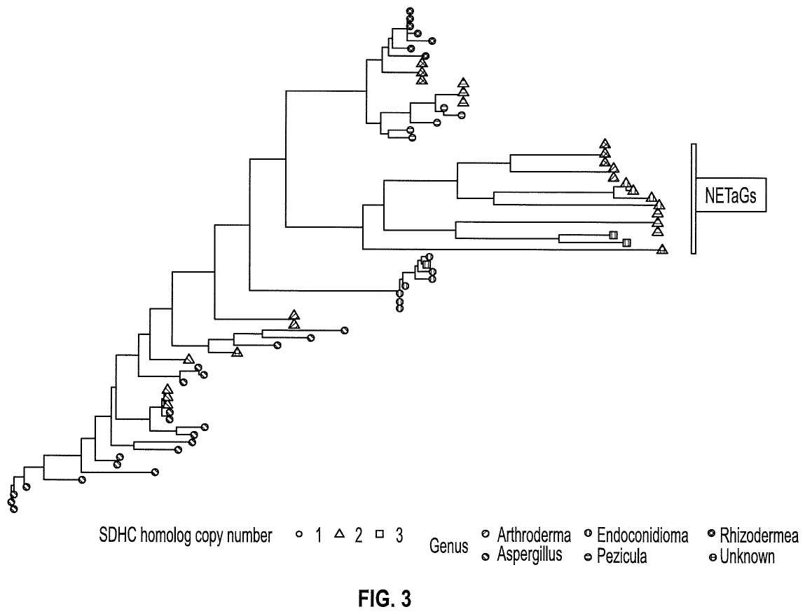

FIG. 3 provides a non-limiting example of a maximum likelihood phylogenetic tree of SDHC homologs from a diverse set of fungal species. NETaGs can be identified by co-localization of homologs from different fungal species and co-localization of single copy and multi-copy homologs in the other branches of the tree.

From the phylogenetic tree, we can infer 14 positive genomes (the genomes containing the NETaGs) and 39 negative genomes (single copy; no NETaG). All genomes are annotated using antiSMASH and the resulting gene clusters are categorized into gene cluster families using the clusteromics approach described above. Resulting families were characterized using the normalized distance:

$$Normalized Distance : \frac{\sqrt{{( TPG - PG )}^{2} + {( 0 - NG )}^{2}}}{\sqrt{{( TPG )}^{2} + {( TNG )}^{2}}}$$

where TPG=the total number of positive genomes, PG=the number of positive genomes in the BGC community, TNG=the total number of negative genomes, and NG=the number of negative genomes in the BGC community, as described above.

Using the number of positive genomes and negative genomes as determined by the phylogenetic tree in FIG. 3 for performing clusteromics, we assume the following: positive genomes (containing the NETaG) contain a BGC that produces a secondary metabolite with activity against the target gene homolog (or product thereof), while negative genomes (only containing the housekeeping-copy of the target gene homolog) do not contain such a BGC. The clusteromics analysis takes all of the BGCs from all selected organisms and returns gene cluster families, which we then test for their presence in positive and negative genomes. We determine the best scoring gene cluster family using normalized distance (see above) as metric. The cluster count shows the number of clusters in the gene cluster family that are potential targets for, in this case, an SDHC inhibitor.

Using normalized distance as a metric, Gene Cluster Family 87 is the best scoring candidate for an SDHC inhibitor among all gene cluster families. The gene cluster family contains mostly two gene clusters per family-which drastically reduces the BGCs that need to be investigated to find a BGC producing an SM with activity against the house-copy. E.g. one species of the genus Rhizodermea contains 90 BGCs. Using the NETaG method combined with clusteromics, we can reduce the number of candidates from 90 gene clusters to just 2 gene clusters from the BGC candidates in the top scoring gene cluster family. Therefore only two BGCs need to be investigated for their activity against SDHC-showing the strength in prediction power of the current invention.

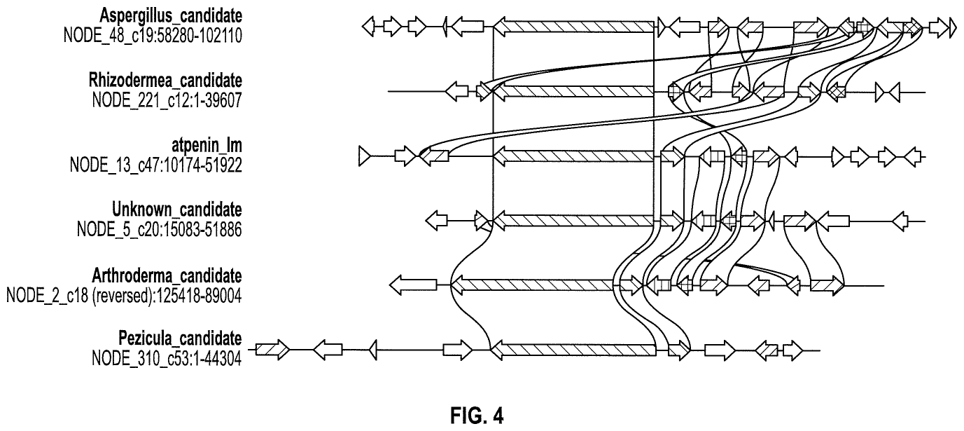

Furthermore, we determined that gene cluster family 87 (see Table 1) contains gene clusters similar to the Atpenin and Harzianopyridone gene clusters (shown in FIG. 4) which are known as potent inhibitors of SDHC. This provides solid evidence that the disclosed methods can successfully predict inhibitors of target genes using NETaGs. The use of NETaGs is not limited to the identification of SDHC inhibitors. The disclosed methods can be used to identify BGCs producing secondary metabolites with functions against any NETaG, and can therefore be used to find new bioactive compounds of interest.

FIG. 4 provides a non-limiting example of a BGC comparison of the Atpenin BGC (a gene cluster extracted using the genomic coordinates from Bat-Erdene, et al. (2020), “Iterative Catalysis in the Biosynthesis of Mitochondrial Complex II Inhibitors Harzianopyridone and Atpenin B”, J. Am. Chem. Soc. 142 (19): 8550-8554) with gene clusters from gene cluster family 87. Each row contains the candidate BGC from the top scoring gene cluster family (see Table 1) for each genus. The arrows depict the genes of the BGC and the shaded area between them shows the sequence alignment between them as produced by the clinker tool (Gilchrist, et al. “Clinker & Clustermap.js: Automatic Generation of Gene Cluster Comparison Figures”, Bioinformatics 37 (16): 2473-2475). The plot shows a great conservation of biosynthetic genes throughout the species, supporting our prediction that the BGC produces Atpenin—a SDHC inhibitor.

TABLE 1
Identifying a new drug candidate through NeTaG mining. Characterizing
Gene Cluster Families by normalized distance.

| Gene | | | | | | | |
|---|---|---|---|---|---|---|---|
| Cluster | Positive | Negative | Genome | Cluster | Normalized | Total | Total |
| Family | Genomes | Genomes | Count | Count | Distance | Positives | Negatives |
| 87 | 13 | 5 | 18 | 25 | 0.12 | 14 | 39 |
| 106 | 6 | 0 | 6 | 6 | 0.19 | 14 | 39 |
| 100 | 6 | 1 | 7 | 9 | 0.19 | 14 | 39 |
| 131 | 6 | 1 | 7 | 7 | 0.19 | 14 | 39 |
| 144 | 6 | 0 | 6 | 7 | 0.19 | 14 | 39 |
| 137 | 6 | 0 | 6 | 6 | 0.19 | 14 | 39 |
| 13 | 6 | 2 | 8 | 8 | 0.2 | 14 | 39 |
| 104 | 6 | 3 | 9 | 9 | 0.21 | 14 | 39 |
| 177 | 6 | 3 | 9 | 10 | 0.21 | 14 | 39 |
| 123 | 6 | 3 | 9 | 9 | 0.21 | 14 | 39 |

### Exemplary Embodiments

Among the embodiments described herein are:
1. A computer-implemented method for identifying resistance genes comprising:
   receiving a selection of at least one target sequence of interest;
   receiving a selection of target genomes from a genomics database, wherein the selection of target genomes comprises a plurality of target genomes from organisms that are known to produce, or are likely to produce, secondary metabolites;
   performing a search to identify homologs of the at least one target sequence in the plurality of target genomes;
   generating a phylogenetic tree based on the identified homologs of the at least one target sequence;
   classifying the genomes of the plurality of target genomes as positive genomes or negative genomes based on the phylogenetic tree, wherein positive genomes are genomes that belong to a clade for which multiple copies of the at least one target sequence homolog are present, wherein negative genomes are genomes that belong to a clade for which a single copy of the at least one target sequence homolog is present; and wherein a target sequence homolog that is present in multiple copies in a positive genome is a putative resistance gene;
   determining, based at least in part on the classification of positive and negative genomes, at least one genomic parameter selected from the following:
      i) one or more scores indicative of co-occurrence of the at least one target sequence homolog (putative resistance gene) and one or more genes associated with a biosynthetic gene cluster (BGC);
      ii) one or more scores indicative of co-evolution of the at least one target sequence homolog (putative resistance gene) and one or more genes associated with a BGC;
      iii) one or more scores indicative of co-regulation of the at least one target sequence homolog (putative resistance gene) with one or more genes associated with a BGC; and
      iv) one or more scores indicative of co-expression of the at least one target sequence homolog (putative resistance gene) with one or more genes associated with a BGC; and
   determining, based on the at least one genomic parameter, a likelihood that the putative resistance gene is a resistance gene.
2. The computer-implemented method of embodiment 1, wherein determining the likelihood that the putative resistance gene is a resistance gene comprises comparing the at least one determined genomic parameter to at least one predetermined threshold.
3. The computer-implemented method of embodiment 1 or embodiment 2, wherein the selection of at least one target sequence of interest is provided as input by a user of a system configured to perform the computer-implemented method.
4. The computer-implemented method of any one of embodiments 1 to 3, wherein the at least one target sequence of interest comprises an amino acid sequence, a nucleotide sequence, or any combination thereof.
5. The computer-implemented method of any one of embodiments 1 to 4, wherein the at least one target sequence of interest comprises a peptide sequence or portion thereof, a protein sequence or portion thereof, a protein domain sequence or portion thereof, a gene sequence or portion thereof, or any combination thereof.
6. The computer-implemented method of any one of embodiments 1 to 5, wherein the at least one target sequence of interest comprises a mammalian sequence, a human sequence, a plant sequence, a fungal sequence, a bacterial sequence, an archaea sequence, a viral sequence, or any combination thereof.
7. The computer-implemented method of any one of embodiments 1 to 6, wherein the at least one target sequence of interest comprises a primary target sequence and one or more related sequences.
8. The computer-implemented method of embodiment 7, wherein the one or more related sequences comprise sequences that are functionally-related to the primary target sequence.
9. The computer-implemented method of embodiment 8, wherein the one or more related sequences comprise sequences that are pathway-related to the primary target sequence.
10. The computer-implemented method of any one of embodiments 1 to 9, wherein the selection of target genomes is provided as input by a user of a system configured to perform the computer-implemented method.
11. The computer-implemented method of any one of embodiments 1 to 10, wherein the plurality of target genomes comprise plant genomes, fungal genomes, bacterial genomes, or any combination thereof.
12. The computer-implemented method of any one of embodiments 1 to 11, wherein the genomics database comprises a public genomics database.
13. The computer-implemented method of any one of embodiments 1 to 12, wherein the genomics database comprises a proprietary genomics database.
14. The computer-implemented method of any one of embodiments 1 to 13, wherein the search to identify homologs of the at least one target sequence comprises identification of homologs based on probabilistic sequence alignment models.
15. The computer-implemented method of embodiment 14, wherein the probabilistic sequence alignment models are profile hidden Markov models (pHMMs).
16. The computer-implemented method of embodiment 14 or embodiment 16, wherein homologs are identified based on a comparison of probabilistic sequence alignment model scores to a predefined threshold.
17. The computer-implemented method of any one of embodiments 1 to 16, wherein the search to identify homologs of the at least one target sequence comprises identification of homologs based on alignment of sequences using a local sequence alignment search tool, calculation of a sequence homology metrics based on the alignments, and comparison of the calculated sequence homology metrics to a predefined threshold.
18. The computer-implemented method of embodiment 17, wherein the local sequence alignment search tool comprises BLAST, DIAMOND, HMMER, Exonerate, or ggsearch.
19. The computer-implemented method of embodiment 17 or embodiment 18, wherein the predefined threshold comprises a threshold for percent sequence identity, percent sequence coverage, E-value, or bitscore value.
20. The computer-implemented method of any one of embodiments 1 to 19, wherein the search to identify homologs of the at least one target sequence comprises identification of homologs based on use of a gene and/or protein domain annotation tool.
21. The computer-implemented method of embodiment 20, wherein the gene and/or protein domain annotation tool comprises InterProScan or EggNOG.
22. The computer-implemented method of any one of embodiments 1 to 21, wherein the generation of phylogenetic trees based on the identified homologs of the at least one target sequence comprises alignment of homolog sequences using an alignment software tool, trimming of the aligned homolog sequences using a sequence trimming software tool, and construction of a phylogenetic tree using phylogenetic tree building software tool.
23. The computer-implemented method of embodiment 22, wherein the alignment software tool comprises MAFFT, MUSCLE, or ClustalW.
24. The computer-implemented method of embodiment 22 or embodiment 23, wherein the sequence trimming software tool comprises trimAI, GBlocks, or ClipKIT.
25. The computer-implemented method of any one of embodiments 22 to 24, wherein the phylogenetic tree building software tool comprises FastTree, IQ-TREE, RAxML, MEGA, MrBayes, BEAST, or PAUP.
26. The computer-implemented method of any one of embodiments 22 to 25, wherein the construction of the phylogenetic tree is based on a maximum likelihood algorithm, parsimony algorithm, neighbor joining algorithm, distance matrix algorithm, or Bayesian inference algorithm.
27. The computer-implemented method of any one of embodiments 1 to 26, wherein the one or more scores indicative of co-occurrence are determined based on identifying positive correlations between the presence of multiple copies of a putative resistance gene and the presence of the one or more genes of a BGC in positive genomes.
28. The computer-implemented method of embodiment 27, wherein identifying the positive correlations between the presence of multiple copies of the putative resistance gene and the presence of the one or more genes of a BGC in positive genomes comprises the use of a clustering algorithm to cluster aligned protein sequences, aligned nucleotide sequences, aligned protein domain sequences, or aligned pHMMs for a group of BGCs to identify BGC communities within the plurality of target genomes.
29. The computer-implemented method of embodiment 27, wherein identifying the positive correlations between the presence of multiple copies of the putative resistance gene and the presence of the one or more genes of a BGC in positive genomes comprises the use of a phylogenetic analysis of protein sequences or protein domains for a group of BGCs to identify BGC communities within the plurality of target genomes.
30. The computer-implemented method of embodiment 27, wherein identifying the positive correlations between the presence of multiple copies of the putative resistance gene and the presence of the one or more genes of a BGC in positive genomes comprises choosing genomes with a specific taxonomy to identify BGC communities within the plurality of target genomes.
31. The computer-implemented method of any one of embodiments 1 to 30, wherein the one or more scores indicative of co-evolution of a putative resistance gene and the one or more genes associated with a BGC are determined based on a co-evolution correlation score, a co-evolution rank score, a co-evolution slope score, or any combination thereof.
32. The computer-implemented method of embodiment 31, wherein the co-evolution correlation score is based on a correlation between pairwise percent sequence identities of a cluster of orthologous groups (COG) for the putative resistance gene and pairwise percent sequence identities of a cluster of orthologous groups (COG) for one of the one or more genes associated with a BGC.
33. The computer-implemented method of embodiment 31, wherein the co-evolution rank score is based on a ranking of a correlation coefficient of a COG that contains one of the one or more genes associated with a BGC in ascending order in relation to a COG that contains the putative resistance gene.
34. The computer-implemented method of embodiment 33, wherein in the case of ties for a distance score, the rank for all COGs in the tie is set equal to a lowest rank in the group.
35. The computer-implemented method of embodiment 31, wherein the co-evolution slope score is based on an orthogonal regression of pairwise percent sequence identities of a COG for the putative resistance gene and pairwise percent sequence identities of a COG for one of the one or more genes associated with a BGC.
36. The computer-implemented method of any one of embodiments 32 to 35, wherein only COGs arising from unique positive genomes that have more than three genes remaining after removing corresponding genes from negative genomes are used to evaluate a co-evolution correlation score, a co-evolution rank score, or a co-evolution slope score.
37. The computer-implemented method of any one of embodiments 1 to 36, wherein the one or more scores indicative of co-regulation are based on DNA motif detection from intergenic sequences of the one or more genes associated with a BGC and the putative resistance gene.
38. The computer-implemented method of any one of embodiments 1 to 37, wherein the one or more scores indicative of co-expression are based on a differential expression analysis and/or a clustering analysis of global transcriptomics data.
39. The computer-implemented method of any one of embodiments 1 to 38, wherein the one or more genes associated with a biosynthetic gene cluster (BGC) comprise an anchor gene, a core synthase gene, a biosynthetic gene, a gene not involved in the biosynthesis of a secondary metabolite produced by the BGC, or any combination thereof.
40. The computer-implemented method of any one of embodiments 1 to 39, wherein the putative resistance gene is a putative embedded target gene (pETaG) or a putative non-embedded target gene (pNETaG).
41. The computer-implemented method of any one of embodiments 1 to 40, wherein the resistance gene is an embedded target gene (ETaG) or a non-embedded target gene (NETaG).
42. A computer-implemented method for predicting a function of a secondary metabolite comprising:
   receiving a selection of at least one target sequence of interest, wherein the at least one target sequence of interest corresponds to a gene sequence associated with a biosynthetic gene cluster (BGC) known to produce the secondary metabolite;
   receiving a selection of target genomes from a genomics database, wherein the selection of target genomes comprises a plurality of target genomes from organisms that are known to produce secondary metabolites;
   performing a search to identify homologs of the at least one target sequence in the plurality of target genomes;
   generating a phylogenetic tree based on the identified homologs of the at least one target sequence;
   classifying the genomes of the plurality of target genomes as positive genomes or negative genomes based on the phylogenetic tree, wherein positive genomes are genomes that belong to a clade for which multiple copies of the at least one target sequence homolog are present, wherein negative genomes are genomes that belong to a clade for which a single copy of the at least one target sequence homolog is present; and wherein a target sequence homolog that is present in multiple copies in a positive genome is a putative resistance gene;
   determining, based at least in part on the classification of positive and negative genomes, at least one genomic parameter selected from the following:
      i) one or more scores indicative of co-occurrence of the at least one target sequence homolog (putative resistance gene) and one or more genes associated with the BGC;
      ii) one or more scores indicative of co-evolution of the at least one target sequence homolog (putative resistance gene) and one or more genes associated with the BGC;
      iii) one or more scores indicative of co-regulation of the at least one target sequence homolog (putative resistance gene) with one or more genes associated with the BGC; and
      iv) one or more scores indicative of co-expression of the at least one target sequence homolog (putative resistance gene) with one or more genes associated with the BGC; and
   determining, based on the at least one genomic parameter, a likelihood that the putative resistance gene is a resistance gene that encodes a protein target that is acted upon by the secondary metabolite.
43. The computer-implemented method of embodiment 42, wherein determining the likelihood that the putative resistance gene is a resistance gene that encodes a protein target that is acted upon by the secondary metabolite comprises comparing the at least one determined genomic parameter to at least one predetermined threshold.
44. The computer-implemented method of embodiment 42 or embodiment 43, wherein the selection of at least one target sequence of interest is provided as input by a user of a system configured to perform the computer-implemented method.
45. The computer-implemented method of any one of embodiments 42 to 44, wherein the at least one target sequence of interest comprises an amino acid sequence, a nucleotide sequence, or any combination thereof.
46. The computer-implemented method of any one of embodiments 42 to 45, wherein the at least one target sequence of interest comprises a peptide sequence or portion thereof, a protein sequence or portion thereof, a protein domain sequence or portion thereof, a gene sequence or portion thereof, or any combination thereof.
47. The computer-implemented method of any one of embodiments 42 to 46, wherein the selection of target genomes is provided as input by a user of a system configured to perform the computer-implemented method.
48. The computer-implemented method of any one of embodiments 42 to 47, wherein the plurality of target genomes comprise plant genomes, fungal genomes, bacterial genomes, or any combination thereof.
49. The computer-implemented method of any one of embodiments 42 to 48, wherein the genomics database comprises a public genomics database or a proprietary genomics database.
50. The computer-implemented method of any one of embodiments 42 to 49, wherein the search to identify homologs of the at least one target sequence comprises identification of homologs based on probabilistic sequence alignment models.
51. The computer-implemented method of embodiment 50, wherein the probabilistic sequence alignment models are profile hidden Markov models (pHMMs).
52. The computer-implemented method of embodiment 50 or embodiment 51, wherein homologs are identified based on a comparison of probabilistic sequence alignment model scores to a predefined threshold.
53. The computer-implemented method of any one of embodiments 42 to 52, wherein the search to identify homologs of the at least one target sequence comprises identification of homologs based on alignment of sequences using a local sequence alignment search tool, calculation of a sequence homology metrics based on the alignments, and comparison of the calculated sequence homology metrics to a predefined threshold.
54. The computer-implemented method of embodiment 53, wherein the predefined threshold comprises a threshold for percent sequence identity, percent sequence coverage, E-value, or bitscore value.
55. The computer-implemented method of any one of embodiments 42 to 54, wherein the search to identify homologs of the at least one target sequence comprises identification of homologs based on use of a gene and/or protein domain annotation tool.
56. The computer-implemented method of any one of embodiments 42 to 55, wherein the generation of phylogenetic trees based on the identified homologs of the at least one target sequence comprises alignment of homolog sequences using an alignment software tool, trimming of the aligned homolog sequences using a sequence trimming software tool, and construction of a phylogenetic tree using phylogenetic tree building software tool.
57. The computer-implemented method of any one of embodiments 42 to 56, wherein the at least one target sequence of interest comprises a known NETaG sequence or core synthase gene sequence.
58. A computer-implemented method for identifying a biosynthetic gene cluster (BGC) that encodes biosynthetic enzymes for producing a secondary metabolite having an activity of interest, the method comprising:
   receiving a selection of at least one target sequence of interest, wherein the at least one target sequence of interest comprises a sequence that encodes a therapeutic target of interest;
   receiving a selection of target genomes from a genomics database, wherein the selection of target genomes comprises a plurality of target genomes from organisms that are known to produce secondary metabolites;
   performing a search to identify homologs of the at least one target sequence in the plurality of target genomes;
   generating a phylogenetic tree based on the identified homologs of the at least one target sequence;
   classifying the genomes of the plurality of target genomes as positive genomes or negative genomes based on the phylogenetic tree, wherein positive genomes are genomes that belong to a clade for which multiple copies of the at least one target sequence homolog are present, wherein negative genomes are genomes that belong to a clade for which a single copy of the at least one target sequence homolog is present; and wherein a target sequence homolog that is present in multiple copies in a positive genome is a putative resistance gene;
   determining, based at least in part on the classification of positive and negative genomes, at least one genomic parameter selected from the following:
      i) one or more scores indicative of co-occurrence of the at least one target sequence homolog (putative resistance gene) and one or more genes associated with a biosynthetic gene cluster (BGC);
      ii) one or more scores indicative of co-evolution of the at least one target sequence homolog (putative resistance) and one or more genes associated with a BGC;
      iii) one or more scores indicative of co-regulation of the at least one target sequence homolog (putative resistance gene) with one or more genes associated with a BGC; and
      iv) one or more scores indicative of co-expression of the at least one target sequence homolog (putative resistance gene) with one or more genes associated with a BGC; and
   determining, based on the at least one genomic parameter, a likelihood that the putative resistance gene is an actual resistance gene associated with a BGC that produces a secondary metabolite that acts upon a protein product encoded by the resistance gene.
59. The computer-implemented method of embodiment 58, wherein determining the likelihood that the putative resistance gene is an actual resistance gene associated with the BGC that produces the secondary metabolite comprises comparing the at least one determined genomic parameter to at least one predetermined threshold.
60. The computer-implemented method of embodiment 58 or embodiment 59, wherein the selection of at least one target sequence of interest is provided as input by a user of a system configured to perform the computer-implemented method.
61. The computer-implemented method of any one of embodiments 58 to 60, wherein the at least one target sequence of interest comprises an amino acid sequence, a nucleotide sequence, or any combination thereof.
62. The computer-implemented method of any one of embodiments 58 to 61, wherein the at least one target sequence of interest comprises a peptide sequence or portion thereof, a protein sequence or portion thereof, a protein domain sequence or portion thereof, a gene sequence or portion thereof, or any combination thereof.
63. The computer-implemented method of any one of embodiments 58 to 62, wherein the selection of target genomes is provided as input by a user of a system configured to perform the computer-implemented method.
64. The computer-implemented method of any one of embodiments 58 to 63, wherein the plurality of target genomes comprise plant genomes, fungal genomes, bacterial genomes, or any combination thereof.
65. The computer-implemented method of any one of embodiments 58 to 64, wherein the genomics database comprises a public genomics database or a proprietary genomics database.
66. The computer-implemented method of any one of embodiments 58 to 65, wherein the search to identify homologs of the at least one target sequence comprises identification of homologs based on probabilistic sequence alignment models.
67. The computer-implemented method of embodiment 66, wherein the probabilistic sequence alignment models are profile hidden Markov models (pHMMs).
68. The computer-implemented method of embodiment 66 or embodiment 67, wherein homologs are identified based on a comparison of probabilistic sequence alignment model scores to a predefined threshold.
69. The computer-implemented method of any one of embodiments 58 to 68, wherein the search to identify homologs of the at least one target sequence comprises identification of homologs based on alignment of sequences using a local sequence alignment search tool, calculation of a sequence homology metrics based on the alignments, and comparison of the calculated sequence homology metrics to a predefined threshold.
70. The computer-implemented method of embodiment 69, wherein the predefined threshold comprises a threshold for percent sequence identity, percent sequence coverage, E-value, or bitscore value.
71. The computer-implemented method of any one of embodiments 58 to 70, wherein the search to identify homologs of the at least one target sequence comprises identification of homologs based on use of a gene and/or protein domain annotation tool.
72. The computer-implemented method of any one of embodiments 58 to 71, wherein the generation of phylogenetic trees based on the identified homologs of the at least one target sequence comprises alignment of homolog sequences using an alignment software tool, trimming of the aligned homolog sequences using a sequence trimming software tool, and construction of a phylogenetic tree using phylogenetic tree building software tool.
73. The computer-implemented method of any one of embodiments 58 to 72, further comprising performing an in vitro assay to test a secondary metabolite produced by the identified BGC for activity against the therapeutic target of interest.
74. The computer-implemented method of any one of embodiments 58 to 73, further comprising performing an in vivo assay to test a secondary metabolite produced by the identified BGC for activity against the therapeutic target of interest.
75. A system comprising:
   one or more processors; and
   a memory communicatively coupled to the one or more processors and configured to store instructions that, when executed by the one or more processors, cause the system to perform the method of any one of embodiments 1 to 74.
76. A non-transitory computer-readable storage medium storing one or more programs, the one or more programs comprising instructions which, when executed by one or more processors of a system, cause the system to perform the method of any one of embodiments 1 to 74.

It should be understood from the foregoing that, while particular implementations of the disclosed methods, devices, and systems have been illustrated and described, various modifications can be made thereto and are contemplated herein. It is also not intended that the invention be limited by the specific examples provided within the specification. While the invention has been described with reference to the aforementioned specification, the descriptions and illustrations of the preferable embodiments herein are not meant to be construed in a limiting sense. Furthermore, it shall be understood that all aspects of the invention are not limited to the specific depictions, configurations or relative proportions set forth herein which depend upon a variety of conditions and variables. Various modifications in form and detail of the embodiments of the invention will be apparent to a person skilled in the art. It is therefore contemplated that the invention shall also cover any such modifications, variations and equivalents.

1. A computer-implemented method for identifying resistance genes comprising:
receiving a selection of at least one target sequence of interest;
receiving a selection of target genomes from a genomics database, wherein the selection of target genomes comprises a plurality of target genomes from organisms that are known to produce, or are likely to produce, secondary metabolites;
performing a search to identify homologs of the at least one target sequence in the plurality of target genomes;
generating a phylogenetic tree based on the identified homologs of the at least one target sequence;
classifying the genomes of the plurality of target genomes as positive genomes or negative genomes based on the phylogenetic tree, wherein positive genomes are genomes that belong to a clade for which multiple copies of the at least one target sequence homolog are present, wherein negative genomes are genomes that belong to a clade for which a single copy of the at least one target sequence homolog is present; and wherein a target sequence homolog that is present in multiple copies in a positive genome is a putative resistance gene;
determining, based at least in part on the classification of positive and negative genomes, at least one genomic parameter selected from the following:
i) one or more scores indicative of co-occurrence of the at least one target sequence homolog (putative resistance gene) and one or more genes associated with a biosynthetic gene cluster (BGC);
ii) one or more scores indicative of co-evolution of the at least one target sequence homolog (putative resistance gene) and one or more genes associated with a BGC;
iii) one or more scores indicative of co-regulation of the at least one target sequence homolog (putative resistance gene) with one or more genes associated with a BGC; and
iv) one or more scores indicative of co-expression of the at least one target sequence homolog (putative resistance gene) with one or more genes associated with a BGC; and
determining, based on the at least one genomic parameter, a likelihood that the putative resistance gene is a resistance gene.

2. The computer-implemented method of claim 1, wherein determining the likelihood that the putative resistance gene is a resistance gene comprises comparing the at least one determined genomic parameter to at least one predetermined threshold.

3. The computer-implemented method of claim 1, wherein the selection of at least one target sequence of interest is provided as input by a user of a system configured to perform the computer-implemented method.

4. The computer-implemented method of claim 1, wherein the at least one target sequence of interest comprises an amino acid sequence, a nucleotide sequence, or any combination thereof.

5. The computer-implemented method of claim 4, wherein the at least one target sequence of interest comprises a peptide sequence or portion thereof, a protein sequence or portion thereof, a protein domain sequence or portion thereof, a gene sequence or portion thereof, or any combination thereof.

6. The computer-implemented method of claim 1, wherein the at least one target sequence of interest comprises a mammalian sequence, a human sequence, a plant sequence, a fungal sequence, a bacterial sequence, an archaea sequence, a viral sequence, or any combination thereof.

7. The computer-implemented method of claim 1, wherein the at least one target sequence of interest comprises a primary target sequence and one or more related sequences.

8. The computer-implemented method of claim 7, wherein the one or more related sequences comprise sequences that are functionally-related to the primary target sequence.

9. The computer-implemented method of claim 8, wherein the one or more related sequences comprise sequences that are pathway-related to the primary target sequence.

10. The computer-implemented method of claim 1, wherein the selection of target genomes is provided as input by a user of a system configured to perform the computer-implemented method.

11. The computer-implemented method of claim 1, wherein the plurality of target genomes comprise plant genomes, fungal genomes, bacterial genomes, or any combination thereof.

12. The computer-implemented method of claim 1, wherein the genomics database comprises a public genomics database.

13. The computer-implemented method of claim 12, wherein the genomics database comprises a proprietary genomics database.

14. The computer-implemented method of claim 1, wherein the search to identify homologs of the at least one target sequence comprises identification of homologs based on probabilistic sequence alignment models.

15. The computer-implemented method of claim 14, wherein the probabilistic sequence alignment models are profile hidden Markov models (pHMMs).

16. The computer-implemented method of claim 15, wherein homologs are identified based on a comparison of probabilistic sequence alignment model scores to a predefined threshold.

17. The computer-implemented method of claim 1, wherein the search to identify homologs of the at least one target sequence comprises identification of homologs based on alignment of sequences using a local sequence alignment search tool, calculation of a sequence homology metrics based on the alignments, and comparison of the calculated sequence homology metrics to a predefined threshold.

18. The computer-implemented method of claim 17, wherein the local sequence alignment search tool comprises BLAST, DIAMOND, HMMER, Exonerate, or ggsearch.

19. The computer-implemented method of claim 17, wherein the predefined threshold comprises a threshold for percent sequence identity, percent sequence coverage, E-value, or bitscore value.

20. The computer-implemented method of claim 1, wherein the search to identify homologs of the at least one target sequence comprises identification of homologs based on use of a gene and/or protein domain annotation tool.

21. The computer-implemented method of claim 20, wherein the gene and/or protein domain annotation tool comprises InterProScan or EggNOG.

22. The computer-implemented method of claim 1, wherein the generation of phylogenetic trees based on the identified homologs of the at least one target sequence comprises alignment of homolog sequences using an alignment software tool, trimming of the aligned homolog sequences using a sequence trimming software tool, and construction of a phylogenetic tree using phylogenetic tree building software tool.

23. The computer-implemented method of claim 22, wherein the alignment software tool comprises MAFFT, MUSCLE, or ClustalW.

24. The computer-implemented method of claim 22, wherein the sequence trimming software tool comprises trimAI, GBlocks, or ClipKIT.

25. The computer-implemented method of claim 22, wherein the phylogenetic tree building software tool comprises FastTree, IQ-TREE, RAxML, MEGA, MrBayes, BEAST, or PAUP.

26. The computer-implemented method of claim 22, wherein the construction of the phylogenetic tree is based on a maximum likelihood algorithm, parsimony algorithm, neighbor joining algorithm, distance matrix algorithm, or Bayesian inference algorithm.

27. The computer-implemented method of claim 1, wherein the one or more scores indicative of co-occurrence are determined based on identifying positive correlations between the presence of multiple copies of a putative resistance gene and the presence of the one or more genes of a BGC in positive genomes.

28. The computer-implemented method of claim 27, wherein identifying the positive correlations between the presence of multiple copies of the putative resistance gene and the presence of the one or more genes of a BGC in positive genomes comprises the use of a clustering algorithm to cluster aligned protein sequences, aligned nucleotide sequences, aligned protein domain sequences, or aligned pHMMs for a group of BGCs to identify BGC communities within the plurality of target genomes.

29. The computer-implemented method of claim 27, wherein identifying the positive correlations between the presence of multiple copies of the putative resistance gene and the presence of the one or more genes of a BGC in positive genomes comprises the use of a phylogenetic analysis of protein sequences or protein domains for a group of BGCs to identify BGC communities within the plurality of target genomes.

30. The computer-implemented method of claim 27, wherein identifying the positive correlations between the presence of multiple copies of the putative resistance gene and the presence of the one or more genes of a BGC in positive genomes comprises choosing genomes with a specific taxonomy to identify BGC communities within the plurality of target genomes.

31. The computer-implemented method of claim 1, wherein the one or more scores indicative of co-evolution of a putative resistance gene and the one or more genes associated with a BGC are determined based on a co-evolution correlation score, a co-evolution rank score, a co-evolution slope score, or any combination thereof.

32. The computer-implemented method of claim 31, wherein the co-evolution correlation score is based on a correlation between pairwise percent sequence identities of a cluster of orthologous groups (COG) for the putative resistance gene and pairwise percent sequence identities of a cluster of orthologous groups (COG) for one of the one or more genes associated with a BGC.

33. The computer-implemented method of claim 31, wherein the co-evolution rank score is based on a ranking of a correlation coefficient of a COG that contains one of the one or more genes associated with a BGC in ascending order in relation to a COG that contains the putative resistance gene.

34. The computer-implemented method of claim 33, wherein in the case of ties for a distance score, the rank for all COGs in the tie is set equal to a lowest rank in the group.

35. The computer-implemented method of claim 31, wherein the co-evolution slope score is based on an orthogonal regression of pairwise percent sequence identities of a COG for the putative resistance gene and pairwise percent sequence identities of a COG for one of the one or more genes associated with a BGC.

36. The computer-implemented method of claim 32, wherein only COGs arising from unique positive genomes that have more than three genes remaining after removing corresponding genes from negative genomes are used to evaluate a co-evolution correlation score, a co-evolution rank score, or a co-evolution slope score.

37. The computer-implemented method of claim 1, wherein the one or more scores indicative of co-regulation are based on DNA motif detection from intergenic sequences of the one or more genes associated with a BGC and the putative resistance gene.

38. The computer-implemented method of claim 1, wherein the one or more scores indicative of co-expression are based on a differential expression analysis and/or a clustering analysis of global transcriptomics data.

39. The computer-implemented method of claim 1, wherein the one or more genes associated with a biosynthetic gene cluster (BGC) comprise an anchor gene, a core synthase gene, a biosynthetic gene, a gene not involved in the biosynthesis of a secondary metabolite produced by the BGC, or any combination thereof.

40. The computer-implemented method of claim 1, wherein the putative resistance gene is a putative embedded target gene (pETaG) or a putative non-embedded target gene (pNETaG).

41. The computer-implemented method of claim 1, wherein the resistance gene is an embedded target gene (ETaG) or a non-embedded target gene (NETaG).

42-72. (canceled)

73. The computer-implemented method of claim 39, further comprising performing an in vitro assay or an in vivo assay to test a secondary metabolite produced by the identified BGC for activity against the therapeutic target of interest.

74. (canceled)

75. A system comprising:
one or more processors; and
a memory communicatively coupled to the one or more processors and configured to store instructions that, when executed by the one or more processors, cause the system to perform the method of claim 1.

76. A non-transitory computer-readable storage medium storing one or more programs, the one or more programs comprising instructions which, when executed by one or more processors of a system, cause the system to perform the method of claim 1.
